SEQUENCE LISTING (62 sequences)

SEQ ID NO: 1 - DNA, 1674 bp, Ceratitis capitata
tcataagtaggagaacacaatttcaatattaattctatccgtgctttcgctacatttatctaaatgcatacacagaaaggcaacgaaagagtaaactttgtgtttttatctgtaagcagcacgcgagaattgaaataattcatcttctgggtcattttattgatttttaaccacttttaaaagaaaccctttaattttatgtaaataaaaatagcgaagccgcacagcaacagtgatgccctaaacacactgacaattactatcatagtgtaaagatatttattcggcatcgcgataacaaatttaacaacgaattgaaagaaataaatatatcaaatttggcatgaaaccactgcaaagtcgtatctcaacaatgttgatcaagcaacgcaattaatatcagttaccgctccaatacaagttctttatttctatggaagattaatacgaaatatgggaagatggatctgaatttatatgaaataaggtttttggacttattcaataacatttattatttaaataatggtcacatttctggtatgtatactttcctactctatttaaaaattaaggatcaaagtcaagtgtagagcggttggttgggtggattgcgtgtttatggttattaaggtttgtctttaccaaaatttttttttttatataaatcccattacctttcgcatcacaatcaacgcaatatttgttgtcttcatcacgtagcatctgagtcagtaaagtttgacatttttcctgtattaatttggtcctttcattttctttgcggctagaagttgagctcatttttacttttctctttttatgaacacacaagaaaaaaactttgtttgccacggtttatatttgcacttgtactacactttttgcttttattttctacaattatgactgctgcatttgtttataaattggaatataattctgtaaattgctaattttgagtgaattccggagaaactgctactgaaataacttcaatactaatctaaaaattaaaatgatttaccactaacatgaccgaaagaaaaacgtcgaatgaaggcgaatatttaagattgtcagaggcaaacggcgaatttaacatgtcattgaagtgtgtagagttttcataaatttggtcgttcacaatgaccacaccagctttgtagagctttggaaaatttactaagggactagggactaggttagcggtgttcactcaaaaacaatagagatatgttctttctcgtagcaacgaacgtatattaaaacattaatatacgttctttggtcaaattactttcaaaaaatttattaacaatagaatagtatatttctataggaaaatccaaaaagaaattcaagtcacaatttccaaatacacatatgtatgtaggccttacaaacatcgcaaatttcagaaacaaaatatattttttaatttatttgtagttgtaagtaattcgcaggtgattgtcgaaatttgtaattgtgaatatgtaagttttttgagtggtaagtgactgtgaactattaccgcaacactttactgtaaatggttcatatcggaacaaagaagagaaataaattcaattttgaatgtgaaacggaatctggctaaaataaacaattttgtgaatatttgaaatacttgtttattgaaaatccaatatg

SEQ ID NO: 2 - DNA, 1516 bp, Ceratitis capitata
catggtgagaaatgggctcggggttgcccatcgatggacgtgatcggagaaaatgtcaagaaagtcgaagaattaaattttgacagatagataccgcgaacaatagagttgtgtctaccgctcgggttatgacgggaagctactgggcttacaggaattgaccatccaccctacagtccggacccggctcccagtgacttttacctttattcaaatttagaaaactactaacggggacgaaaattttcagacaaaagcggccgtagaaaaccatttagaagagaaatctaaagaatatattttcagtggtcactcatcatccttcaaaatgtaattagttgaaaattagaccaagaaaacaaatgagaaataggttttgacatttcgattgttcgttttcacaaataatatacattgagggtaaattgggtaaattcgccttggtgaaagtgtactttaacttacgttgactttcaaatcgataaattttgaaaataattggcgctactgtgcaaggcgaagccggtgttggctcgtgttggctcagggttacaatccaaaacgcagcggaaggccaccaacttacaaagaagtttaactttacttggaatttttaggtgaataacattttgattttttaaaaaaatttctattttatttttctgcattttaattcttttttattattatttttttactgagtggccacgcctgcctagacaacatactgttccgattttgctgaataaacctcgatgattttctgaatccttcgggtacgatttctggattgttaaataaaaaatcgccaacttaattggcagtttctttcgttataaagaatttatctaatgtcctttatttactcagtaattcacaattcaattatactgcaattcactagttacggttaaggtattattttacttagtagtctggcaggaccaaagtggacgtaattttgcttttatgactaaaaatttaaaataaaattattaaagagctatgagccagctagggaaaagaaaagcagttctagaatctaggctttttttctatttccatacatatagaattcatttacgaaagcctgcggctgaaaactaaaatttcccaaaaagattttttatttgtttaattattaatagaagttgaataaattctttacgaattgtgcagtgatcgaactcgaagaatttgggaaatcattgaggcgtatttagtaaaatcctcgcaataatgttagcataaattcgagttcagtattatcgcataaactggatgtggcacgccttaaaacaaaactttaataattctaagctggcagcactgtttcgttttatgtttttaaaaaacatccccagtcccaaaacaacgtgctattcctttcttgcctgtgtgaaataaaagcaaacgtcaaaggcagatagagattattgaattgcgcatacttggtgtacttggtgcgtttggtcaagtgtgtgtgagcgtggtaaagtgttcaattatcaaaatg

SEQ ID NO: 3 - DNA, 1933 bp, Ceratitis capitata
ctaggtagtattccttattgaccgtacgaccttgtggcaagaattcatgatgcaccacgccatttctgcagaggtaaacagtgagcaatactttgacatctgacagaacttgacgtgctcaggcgaaagtcattggcaacttctcggccttccttaaaccggttatatcactgttgaacttgtattctgctcatagtaaactctgcaaaagtaacagtcaatattttttacacaaaactttaacagtttttgatccattttttcaaatagtaaaaaatcgacaaggacacctaaatacggataaccgttttatatgtcaaaaacatataaacaatccaaaatggctgatattgccaacagatgtaccaacacaacgaaaaaaaaacgaaaatcgaaaatacgtaactcaggaaaatttgaaaatagcggatatgacctcgtacatacctaaattaatgggaaattatagtttttctcacctgtaagacacttttgattccgaaaaaccttctcgttggaaaaaatcagttatcataaagaagaattaacttcaagaattttgtaaatagagttttttttggggaaactgggaaaactgaaagttataaatgctgcattgaaattaattatggtgatttatggtacatcttttaaagaaaagtaagcaaataaaatattttcagaaacaaaaaagaatttgtatttttctgtgaaaaaaatgtaagcccatggatcagagtcggtcaaaggtcaagcttaaagatatttaatccaggcttccgattttcgcaaggtggccttcagtgaaaagaattaattaataataagtgtagttaacgattcattaaaattttcaatgtttaaattccatttccgtttaaaaattttgtttttaaaaataattttataaaattaatttagtttgtcaaaaagaatttgaatagttatacatatatgtctgtaattaattatactttagcctgtaatttgcatattttctttgaatgacctaatatttatctatgtacatatgtatgtgtaacgtgtatgtatgcatttatagcgagtatatataaatgcatgtatgtatatacatatgtatgtatgtatgtatgtatgtatgtatgtatgtatgtatgtatgtatgtatgtatgtatgtatgtatgtatgtatgtatgcatgcatgcatggtcacttgttaaccgagtattcaaggtcgaagggattcacaaatttataaaaaattgtaggcgttgctgcgcaaataacgcatatttgtaacaattgaccaaatgaatgtatcaaaaaagcgtaacaaaagcaattttcttagaaaaaccctaaacactaagtgcagtgaactcaaacacgtatgcaagagtatacatgcatatatttatatgtacacactcgtatgtacaaatgcatatgtgtttttgtatgaatatacatacatatgtgcatttgcaaattatcatagctgaaatgacctattatttgtattattgttaagatttactgttaggcttgcatgcattgtgttccttttgccgcagcattgtcccttcgttgccaggcaagacatcgcgggcttcgttgccggtactcgcgtttgcatgtgcgtgtgtatatgcacataaggatattatgcggattatgaacattttcgcgagagcatttcctggaatttccgtccaaaatcgcgagcattgtttacaaatacatatttttatttgttgtttgtagggattagtgcaatctttcctcggcatgcatatgtccttcttcgtgcatgtatttaactgctgtgtgtgtacgataacttatatgctatccttttctaactttggcttaaattatatgtgatcctttcgtactgatctttccgagagcgtataaaaggcactgccaacagctccgccggccagtatg

SEQ ID NO: 4 - DNA, 661 bp, Ceratitis capitata
catatgtatgcataagtcgtagaaaggaaaatttgggatttagtaggcggaacacttccactttattttccatgaaaaattttaaattttaaaatgctttttcgctaatatttgaccttctgcaaattcgcttttgtttaatcacaaacaaaaaagaaataaatacagacacaaacattttcaaaaacaagctacacaaaagcgattgagtattttttgggtgttactgaagttttactttgtacactaacaaaaaaaaaaaaagaaaactaacaaaattttcacaaaacaatagcagagagaattttaaattcacgaatcttagcactagagattagtggatacgcaggattagaaaatattttgaaaattatatacagacgtgtttcgtcgattatttgctctgcgctgcttggtaagtaacaaaaattgagacaataagatgctcacaggtaaagtgcgaaaaagcctactactcctatttttcatccaaaatgtcttcactcaaattattgtgctcacgaaatataaaaggaaaaaatcttccatacttggaacagtcaagcttggtgcttagcagatgcattaagtcaacttcagagcaacacaaaatttcgttcggtttacaatcgctgagatcagtaacatcatcacagaaatg

SEQ ID NO: 5 - DNA, 1405 bp, Ceratitis capitata
aacgtttcctgctcgtttgtttaggttagtttttcgctttgaaagttttagttttattattacatatttatttccattaaaaaataaataaataaataaataaacttaaataaatatttctgtggaaatatttgtgtgaaaaaaagagataattaaatttttttgttaattttcttaataaaacatcaaaaaacgaataaaatattaaacattattaataatatgattattctttaaattcaattcatgtaagtttcgtattaatttcagaaaattctgaaaactttctcgactagcttatgtccattttctcaagttcggacagaaacccgtaaattatgtcaccataatatttatgacgtattttagcatttctttccaccatatgtttcgattttatgctcaagaaatcaaatatttccgagaaagccattcgtttcgtttaggtcgctcgtttacatttcttgcttcacaagtattttccttatcgtagtgttgccaactacttacacttataacaaatatgatttaattgttcacaaaattatgtttctctaacagcactctagagtatggttctatatttataaagaaatttaaaagaaaatgtatataataaaaaaaaaataatcaaaagggaaattgttcaaaaagagatattttttattacctaaccttcaataacaagacactcaaaaatgattgctctcaaaattatgcagtgtttttaagcgcaaaggacgcgaaaagtaaggttcaaaaccaatgattaggaaagtgagtgagagaaagtgaattagagagatatagaaagagagtgtgagatataggcagccatgagagatacagacaaagtagtagagatagaaacagagcaaaaatattcgctacgatttagtacattaacaatcagagctggacgtttgcgagaaagggataaaactatttcctttatatttcactatatcagcgaacgcattttcagcaattacagtaaacaaagcaactattgagataatgacctttcttgagtgctaaaatttgtattaaatttttcaatggaatttggcaaaaacaagtttttatatgatatacctgcaagtagccttcgcttctacgcaagcaattctcgtatgtagcctctcagtcctttcaactcagtgtgcggtttgcgctcacatatttcgtttcgaaaaaattgcttgcctataaattttgactcaaaaatttaaatgcctacctggctatgacaggcatggaaattttccatcctcacaaccatactggcaaaaaggatgatagctcgtgctcgtgagcatgacaagccattcattgtcacataagtgttactctaggtcagactacaaagtgttggaattgtgaaaattaaaaaaaaaaaaaaatg

SEQ ID NO: 6 - DNA, 1273 bp, Ceratitis capitata
attcgcaaagtatacgatcaagtgtgaagaacacaattccagtcactgaagtagatttaaagatttattgaaatacaaaatcgaaaacgaatacaaattgcacacaatggctacacaaaaggacgcaagcttttcctctatacccgtattctcatcgtcatcagagtttcgtgttaacgcctcacacttcagcggagaggatctaaactcgttcgtcgaaatcttagagataatagccaaattgcagcgggaacaacgtgaaaatgaagagcgccgcaatgagcagatagaagaaactcgcttacttctacaaacatcattccatatatcggaaccttcactcaactcactccgctgtgctccgagtccaatatcatcgcctactaaagagaggcttagtctgtgcatctacaaaaatgaaccaggcgctgtgtcaccacaattattaccacctacaacaccgctaccaaagcgacccatttcgcaacgctcccgttcatcagatgaggatgagacttatttcagtgatgtcagcatcagattacaacgcacaccatgccgtacaccgactacattgccaaagaaaccgcgtctcttgtttccgccttcaccagaagcgttgaaaagcgccaaaaatggctcggattccgaagagtccggcatataccctctagctaatagttcatcaacatcagcgcaaaaagtgccgaagacaatacgcgcgcgcaagcgtcttatgagtttcgagaaacccaaacgcaccatacgcttacgcatactcgatttactactgagacgagatcatctgcatgagaggaagatcgctaccaatggtagaaatacaaaaattgcattttccacattggatttgagcgtcgatcgagcccaaaatgagaaatacagtaatttagctttgcgtttgttgcgcaaaatgaagaaagagttcacgacaaaggaggtgggcaatgaaaatcgggcggaattagagaaattggacaaagcattgagctatatgactttgtaatgaccgaccctgaagaagaagatacagattaccaaaatgatctccatgagattgaaatatctaacggtatgaatgacgttcgatgaactgtaagagcgagtgaaatggatatggaagtgggctacaaatttagttttaagtacataatttattacattgttagttataaccactcattagttgcatatatgtactttaataaatcattttttattaataaagtatatgacaaaaaaaaaaaaaaaaaaa

SEQ ID NO: 7 - DNA, 2526 bp, Ceratitis capitata
  5′UTR (1)..(163)
  3′UTR (2147)..(2526)
gagtggtaagtgactgtgaactattaccgcaacactttactgtaaatggttcatatcggaacaaagaagagaaataaattcaattttgaatgtgaaacggaactctggctaaaaataaacaattttgtgaatatttgaaatacttgtttattgaaaatccaatatgacggcggacggatcacaggagctgaagtgccaactgatgaaatgtagagaagttatatctgttggatataacacagaaagtcgaattgaatggttaaataacttctgtggttacttttatgagttcgccgatcgtctgcataaatatatctccaccgaagtcacaggggatctgtcagggaatgaacatatagatatcacttttctttgcctagctcaggtgtgcctgtgtacaaaatatttggaacgtgtcatacgagctgaggaaactgccggcaaaccaataccatcttctcgaacgcattttatagatcggataaatttatgtttagacaaattagtactatcaacgaggaacttagaagtcacaggagatgaagaaaaagctcttcaacagtcgcgatatccttttttcgacttattagaaatagccatggatcatattaagaactacagtaattaccaagaaagtctgaatgatgaagtagataaatctcgcgagcttaaagatgcatttgactccagcaaagaagttcatcaatgcgttcgtttcatgattagtcaggcacttgctttggcaaatgtagcgttaattgaagataaaacagctatcagtgcattttctcaaaaggttctgcaagatagcactgcatttcaacaggaatgccaaaaaaattttgaaactggtcgaaacaacgaatcgattcgcagcctcaaggcattagcattagcaggatcaataaacaaattacataaacatgtcgatgaaacggttttgcgtttaatatttatttgtttctcagatttagaaaaattttctttggataagcttcgtgccaaaattcataaaaaaagaaaagatgatgcggaactagatgagttcatagcagactttgatgtaaatatggatagattggcgcaaataggattgtttgcttcgaatgatacaaataaaccgaaattaaagacgttagtgcgcagttgtatggcatcacttgaagcactcgattcttgcattataccttcactgcaagcatctaaactcactgctatgcattctgaaattttggagcagcatttctatgaggaaataaagaaactgaaaacagctatttttgatatcgtagacgctgcaccgctcattagaagctattttgatttgctgaattcttgtgtagctaaaatagaaaaaaatttcaacaaatcccaattagatgatatacttcaaatgggtgaatttgttttacaattctttcaatatcccagtaataaaaaaatacttcaaccgacgcagaagttaatatacaatcgcctagaacattttcaaaaatacaaattaatgttgaaggaatgtcgtgctattttggtatgtgctggccaggtggatcacaaacgtatagtaaaacgatttaaaatattacgcggcataatgcaaaggttcgtagatgcacttgaatatgaatataaacaaaatgagcctacgaaacgaattgaaccaaaccttacatgggataaacttgctgttggcgaaaatttaactatcgatttaaatgaaacacaatttcaacttgagagtatagaaccgagcatatgtagtatactttaccgcaatgagtccgatatttttaaatcacgtaagcgtgctctaagcgaatcaatgtatggtaataactcaaggtgttcgcctacaacaagccaagaaatgcgggataaagaaaagcagaatttacaaagaagatttagcgctgatggtccaaataagcaagagcgtagtagagtcagcttgcgtagaaaggaaagtttgcgcacaacaatgtttaaacgccaaaaatctctggaatcaagaaaagcctgcaatttttatttacaaaattctgccagtttgcaaatatcagaaatactcgatcaaatttcggaaatatcgtcgaacaactcgatacactcgggcgaagatgtactcaataaaactcgcattgggtaacttttgcaatgtggagttacaccattgcagttgaaaagggattttaaaaaatttatatacggaatttctgcactgtaatgtaacatataattatcttttaaaaacctctgtttatactttagtttttacttgtgaaatgctttaatggtttaaagaatatcttttgtattattgtgatctctttacttgtttactatgtcctataaagttttttcaaataattttgtatttgtaagcacataattgtatatccgtatacatcagtttatgaaatattttttattatgaatcccttattttgcaacctcactgtttttatacgcatatgtatgtaataaaatcacttttaaagcaaaaaaaaaaaaaaaaaaaaaaaaaaa

SEQ ID NO: 8 - DNA, 3329 bp, Ceratitis capitata
  5′UTR (1)..(9)
  3′UTR (3298)..(3329)
attatcaaaatgtctgacttgaatttgaatgacgacttattgaattatgaattaggtgatgatatcgatgatcaaacactttatggggcagatgaggatgaattgctattgtctgacgatgagttggaaaaagatgttacaaaagaagttaaacaaaaaataaaagcagaggcagaacaatgggttaatgaacgtattcaggaaaaagaagaacatcaaaatgtttcaaaacaaactaaaacatccagtttacaaatatcaggaactgttggcattaaacatgctgcgaaatcaatcaaagataccgccaaaccagcactggcagacgcaacagaggtaacttctacagcggcgtctcatgcgttggcaattgtcccgcaacatggtgatgtaacagatgacgtctctaccagtgagcattcgatagccacgcatgtcgaattcaaagaacccgcaaaatcgcaaacacagaaagaattaatacagcacttacccccagtatctactcaaacagttttacaaaatgccacaaatatcgacgaagtgacatcaagcagcagcaaagaggcggaaatagatcactcgtcagttatcagctgtagtactggcaatctaacagattcatcctttatacaaagctctcaggagagcttgggattggctacacttagcgccagtgacttctccgatcctagagaggatatggaagaggagcgtgagacgagaaccaatataaagacaacaaatgaacgagatttagatacaggaatgaatgaatcggcagataaacaccattcccgttatgtgcacaataagcgtggtggaaaccctctgctgagaccccattcgattcgtggctcttaccatggtcaaaggccactcctgcgatatccgccatcacatggtcaattcccgttacaacaaccgtccttaccttacgcaccacacaacacagtacacggtccaccatcaactaatcctggaaccatcggcccaccacagacaaaccccaatgcaccgattcctctccatccacacgatcccatgaatcccaccatctcccatcgaccccgacatccatatcctggctttcgcccaagtggacctatgaattttcgccacccaaatccagcgaatatgtatcatcatgattataatacaggcgcaggaataccttttaatcagaacttcggacctatgccacatccagcacgacctatgttccgaccagaactcggagggggacctatgacaggaggcaacaatgtaggtctgggaccaactgggggacccatgggaatgaggccaaacatgcgcctaccacgaccacctctgtcaaatttagcgccgccaaactttatgaataatcctggaatcaggccaccaacacaacaacaacaacagccattgcctcagcaaccaggtcctctgcaggcaccacaaaatgtcggcgtagtaccagtgcaagcgcctacaagtggtagtggtggccctttgccatcgactgcagtgcgtccgagtaaagtgctgatcaatcctaacttcaaaggaggcgtacaagcagctacgaacaagtttataaaagaaactcagtttatgtccacaattagcagtcatgtctctcacctacagagtgacgatgaattgttgcgacaacaagaggaattcatcaataaaaatcgagaacacattgagaaacgtcgacatgaacgctcaccgctcgcctcgcgacgcagccgtagtcgcagccgttcacgaacactttcacgtgagcgaagtttttcaccaccaaataaaagaggtggcgcaggcagtggcagtggaagtgccggcggaccggctagtttacatttcgacagagaacgcgagcgtgaccgcgaacgagaccgagaacgtgatcgcgatcgggaacgagatagagatcgtttagatcgtgaaagagatcgcgatcgtgaaagggaaagagaacgtggtcctatcgaccgagaacgtggtgttttgggtagcggaaattctaacctaggaccaagaggaaatcgtttccggcgtgctaacagtcgagatcacgattatgataaaagagctgctggtagttatattaaacggaggcgcagtttatcgcctctaccacgagctggaggcggctttcgacgtggtggcgatcgcgaacgcccagatgttgaagaggatgaagaaacacgtgtttataggttggaaattgagaaacagaaagcattacgggaaaaaatattgaaggacaaagaattaaagcgccggcgcgcagccgaagaaaaacagtatgaggatcatcgccccactcaacctaataataacagtaacaacggcagcaacaacagtagtaattctccaggcaatgctactgagaaccaaacgcagaagcttaaaccgattgtggtcacagagcgcaagataatatcattgaagaagcggcaacaacaacattattctgatgaagaactaccacggcaaaaaccacaacaacaagaccaacatcagcgtacacaactgtcacactcgcagacacaaaatcaacgcaaactaattccaactgcaaaaattctaccggaagccaaacgaaacctaaatgatcatataacgaatgttacaagtagtagcagtgctagtggtggcagcgctggtatcagcagtggcggtgcgaatagtggcaatagcaacactgctataataaaaccggtgcaaaagaaagacacacacacaccaacaccgccaccggcagctatattgaagcaaacacgtcgtatgtcctcaccaagtgatgatgaagttgaacttgactacgatgaagatgaactaatgcttgaaatggaggatcgtctgttggccacaccaacaccatccccaccgggtaatgctgtgtcacgtacgcctacacccgaaccaatagcaccgccgcctagtaaggcaaatatgcgatctagtagtcgcagttgtaataataattcaaattcgcaaagatcatcatcatcatcggcaccattcagttcaagtaatcgacgagtcgtattaaaatcatcatcaaacacatctggaggtcgtaaggatagcgatttgcactcatccgagagcagtcgcaatcgtcgcgcgggcggcggaggaggctcatcatcgcgcagcggtggcagtagtaattctaggaagggcatatttgatcgcttggacaccaggcgtcaaatgtatgagagcggcggcaacaacagagatggcggtgatggaaaacgtaacaaaggtcaacgcattgtattaaaacatgattaatacaagtttgaaaataaaaaaaaaaaaaaaaa

SEQ ID NO: 9 - DNA, 5781 bp, Ceratitis capitata
  5′UTR (1)..(3)
  3′UTR (5617)..(5781)
agtatgaatgaacatttcattaaaagtgaacacgcgtatcaagaatatatttatcatcgtgaattgcaaagaatccgaaaacctaatagcataaacagcaataatatgttgctcaataccattgctggcaccagcagtgatctgccaaatacatcacaacaaccacttgtgctcaacacatctaccgtgaacaaagagcatttggaggattgtttaaccatttcgaaggagatcgaacagcgagaggagttgcaaaaagcgtatcgccgccacagccacccgacgccattggaggccacaatgatacagcagcaaatcttcgagtgctccgttgaagatgatagctatttactcgcgctcgaatatcagaattcaaataagccagtacaagtaactgatctagatgccgtttcacagaccatggacgtttgcttgaaatacttcgatgaggatgttaatgtatcgtccggtggatcatgttgtgtaacaccaacaaccagattacctgtcaccggttctgttgccgcagcagtcgccaattatcaaggatatatgaaatatataggttcaccgccgccaccgtacgaagagtgtgtatcgcccaaatcgcgtagtgaacccgaaccgcattctaataacatacgagagttgcttaagaaatacgaacaaaatattgggaaagaaaactcgccgaaaacacaagagaagcagccaaaactccaagaacgtgcatcaaaatcacaaaaacctacgaaattagtagaggagggggttgatcctcaaaaggacgctgtagaagaacaagaaaattcgttgaagccccaagaggccgtgaaaatggaagagcagaaaaaggaggctgatgacgcaccgaacatacgaaacaaatctcttagcccccaagatgatattttaagacgagacgaagagccgtcaaggaaatcatttaatccacaaggggaagaaaaaaagacgtcaaagtctctaaacaaatcgccaagcaaccaacagcatcaaatgaagctccaagaggaggtttcgaaggtatgtaaaccccaagtagtagaagaaaagacatcaaagtcccaaagattatatccaaatctcaaagagatgatgctagagcattctgagttacatccaaagcttcaagaggcatcgccaaagcttacagaaaaatcaccaaagcaggcaagaaatggaaaggacgtgctacgatatgccccgccagtcatactagcagctgagccaacgaaatcgccaaaactttcacgcaaatcgaaagtcaaacaaatggcacatatgtttaataacagaattcatcaactgatgggacggaattcgaacgaaaaaaaaacgagtaacaaagcagatgagaaaaaatcattgaagctaaaaacggatagcaaaccaacggcggcaccaaaaggaaagactcgcttacctacctttcgtactggtctgcccagtccagttccgagtccacgtttcaggcgcaagactacgccagctacaacaacaaaggcaacaccatcacctttcgcacaaaacgagaacgcatgtctcgttgcagaggatgtgttccgtaagctttctgtcaaggacaaggcgctcttgtataataaattcattgaagacatgtctaagcagcatccacaatttagcatccacgcacgtgttgtggaggctacagtgcggaacgagttggaacgcagcgggcatgaagtgacatcaccgaaagttaaagacatggcacgagaactggagtacaaactgcaagcaaccaagccgaaaatgtcgaaatgggctgtcaagcgtctggccttcttgcaaaagactccagaatatgatgcagccagaacacgcgttgtgtctactctgccgggtgaaaactctgcagatgaaagtccaataaaagcgaaagtagatctcgcacaatacatagatgatgaagaatatgaggaaatcgatttagatgacgtagaggacgtagagatcagcacactgacagttgttttgaagcccatgggcgttcgccgagaggagccacgcccacttccgcgcaagcaacgtgagcaaaagttcatcaatgatatgcgcatgatcgaactaacgcagaaacgttacaatgactccatgcgcaagtcggtttccttcgatacaagtttgccaccaaagaagatacgacgcaccagaattgaacgcttgcaacccaaggctatctttcaagatcgatacatggaacgtatgttctacaactggataatggagaagaacggtgtgaccttcaacatcacttcggtagagaatagcgaggactctttgtcgtctaacgttacaaatgaagtgaacacctcaaaaagctcaacgaaatcaaaatcaaaaatgcatgctttactgcataaagcagtagcgaagttagaaagcgttgaggctcgtgtaatgcgacgtgagagtcatcgtctaaagagaatggaaaaggatatgaaaataattaaggagataagggaagatcatgcagctgataaaagtgtaattgaagttgagctgatgaatagcaagacaggcacatcgagtgacgagttgctggacgaacaaaaccggtgtccacaaagaaaaattaaacgtcaggcgccgcaacctccagcagctgaagctaaagtttctctaactgaggaagctgggcagggggtttcacaaagaaaagtgaagcgccaggcgccgcagccgccagctgcggaaacggcagctgctgtaaataatgtcatagaagcaaccgaacaggaggtggagaagaaggtttcacaaaggaaagtgaaacctcaggcaccacagcctcccgcagaatttaataagaacgagatatcagctatggaggagatacaagacacaaaaacccaacagaaggagattttgtctaagccatcgacagccacaaatcccacggaaatactcatcgatgatggggtacaagaaatttcacaaaggaaggtgcaacaccaggtaccacagccaccaacagcaatgggtcaaagcgagccagcaactcaggaggaactgcatgactctaaggctcaaagcgaagagaagaaggttctacaaaaaaaggttaaacgtcaggcaccacagccgccaacagccaccgtaaatctcaacgaaacatcaaatcctgaggaagaaatgtcttcattgtcttctttaggaaagcaatcggaagcggaaagtgaatcgggactctcaagcatactcaagacaagctatgagtcaaataccgaacgaataacacaatccgcaactaaatcagtatcattcgatttgagtcaatctaataaattgacacgtaagaagaagcttcgacggacactttcctggaaaaaagagaattgtgctatggaaggccgcacggtacccacatcaacggactccgaggagtctgaggataaggcacgcgacgacaaaggcgaagaggagaaggaagtagaaaagaaggatgaagagaagaatgaagaggtacaggaaattaaagaagaggagaagggagataacgtcgcttgcgttaatgtgcctacaacagaaccttcttcagagccaaaatttacactgcaacaattatatgaactggccgctgcagcacgagaagcgatcgaaaaacaacaagccccaatcacaccgcagaagataaaatccgcttacacactcacaaccttgccgacctcattattacaaactgaaagcaatctcttgcctacacctttgccacaagccgatccgaatttacagcggcgtcttagcatgtacaaacaaagttcgctaagctcaccaacacttgttttggaacaacaacgtcaaatgcagaaaacacctgagacgccgaaacaacagaaaatccaaaatcgtgagccagagaacacaatgcaacagctgatcgatagttttgtagatctcggttttgagaccggttcaaatggaatggaaagcccaccagtacgtcgtgcaacaggagcgcctccagaaccattgcataggagcacgccacttttcgttagactcacttccgaaatggagcaaagtccacaacaaaatttaacaaattgttcctcaacaccgatcaacaacatgtatcacaacctgccacagccgcatgaggtttttaagagcaatgcagcacgtcagatctttcaacaatcaatcagccctgtatgtgcattcaacgcaagacgtcgccgtacgtcgttaactgaggaaggacgccgcagttctcttgctatgcaagttatatccgaagatcatccgctggagcaagttgaacgtacacaaacgcccttaacacccaacaccggcgctgatgtgagccatattggcaactcattcaattccaacgcaagcagcggcaataacatcgagcgtacagtgcaagagcctacatccaaattttgggtacgtgtcggtgactttaccatttctttggacatcgcctaccatgaaccacaacgtatacgtgctttatacaatatattctctcagaaaagctgtgaggcatatgatttgcactttggcattgacgattacaaattcttcgtagacaagccgaacggcaagacatgtatcacgaagagtttacccaaaagtcaaggcctctcacactattggttctgctcgggtgatctggcaatgccatttaagggcaaatatttagctcctgagaagattgaacgaatgttcgctttcctcaaggacaacgtaaaattcaagatgcagatacaatttggcgtcgatgatatcgagttttcacgcgtgcccgacaagcagcagttcatttcgaaattctcaatggaaagcagctatagcatgcttttgggtctacagggtgcaacacctgttttggatgaacgtaatcgctgtggttggcctaattccggctacggctcggcaaattcgaatacaagctctgtaagagcaggtgatttggattactcggaaaccggttcagatttggataccagtgagatacgtaacatcactggttccccctcaggtaccagttgctattacgatgcagcctcagtgcttagtgccgatggaggctcgatttggccacgtgttagtagcaaaatttcacgtgataattatctcaaaactaaagacgacaacgaggagttggataaactctatgaaaatgaacaaaaacaatatgcacaaagcaagaagtcacacaataacaagcccacacaaagcatgccagaaatgttggaacgcttactggtacagaaggtgcagttggtggctctggaggatcgtatgcgtaaatattcaaatcactcgctgatcgctaaaactcgaatagcagtagccaaggaaataccatatcacatgcgtaagatacgtgccgtaattaatgccatcgacgatattggacaggaaaagggtttcaaaggttatagtctagaaaatttggagggcttcatgtacttcctaatgcgtcaagctgacgtatgttatagcagatgtaacgagcaattgcatgcggttcttgatgccctgattacttatcaacaaattaatcaccaggaattggttaaagctaacaagatgctgggtgtggagactctgaattaatggtgttcaatttcttaaatattatatattaaattatgtacctcaccttgactggtattattgtttgtgtgttgtattgattattttgttaacgttcatttacatttatttcttaaactcaaataaaatgccgacaaggctaaaaccaaaaaaaaaaaaaaaaaa

SEQ ID NO: 10 - DNA, 1876 bp, Ceratitis capitata
  5′UTR (1)..(100)
agtcaagcttggtgcttagcagatgcattaagtcaacttcagagcaacacaaaatttcgttcggtttacaatcgctgagatcagtaacatcatcacagaaatggctaaaacagaaggacagatcaaacgtaaacgtaaaagtgccggacatacaacaacctctcgcgcttcggccgcaaaaagcaaggagttaagtgtattataccaaaattacttgaaatggaaaagtaatttcgaaaagtatgtggcgccgtccacatcacgctctatatcaccaacaaccaacgatatgaatttgaaatgtgtaattgctggtcggtcgacaccgcatcgaaaagaaatcagtcttttacatatatctaaaccatgggacggttgtgaaaccgtaaaacaggaatacatcgaagaaagggactcagatgacctgcaacacttcaacgaaaaaagtgactcggatgaatcagaacgccccgacgaagaaaatgattcacacaactcggaagagctgtctatagaagaaagtgacccagatgagcccgaagaatctttcgaagaaattgattccgatgactcggagcactacacaaccgcatcggagagtggcgagtacagtgtagattgctttaatgcaaatacatttggcgatacttcatatgactgtgaagacgcacattatgtgacagcggacaattttgaatacattaaaagtcatgacagcaactcatccgacgccagtgaagaaccaacagattccagagaatcaaagatcaatttaagcacctacagcatcatttgggacgacgaaataacagtgttaagtgatgtaagtgcttccaagataattttatctgcagaagatcagctgagttctgatagtgaaaattgggtcaacataagcaacgatcaacactccaacgataaatgtgatttaaatcaatcgggacaccccgtcgaagaagaagacaagtcggagcaccccatcgaagaaaattactcggataacaagcaacactccaaagaaggaaatagcgtcgatgactcaaaacacttcatcgaaggaagcaactcggagcactccatcgaacaaaatgattctcatgactcggaagaatcgtctatcgaaggaagtgattcagatgaggccgaacaatctatggaagaaaatgagtcacacgagttggaacaatctattaaagaagaagattcggatgactcacaacacgtcatcgcagaaaacgactcgaagcactccatcgaaggaaatcattcacatgattcggaagagacgtctattgaaggaagtgattcggatgaggccgagcaatctatggaagaaaatgattcacatgattcggaagaggcatccgttgaaggaagtgattcaggtgatgccgagcaatctatagaagaaaatgagtcgcatgattcggaagaatcgtctatcgaaggaagtgattcagatgaggccgaacaatctatggaagaaaatgagtcacacgagtcggaacagtcgtttatcgaagaaggtgacatggaacagtctattgaagaaagtgattctaatgactcagaacattctatggaagaaaatgaatcgcatgattcgaaaaagtcgcccatcaaagaaagtgacttggaacagtctatcgaagaaaatgacccatatgagtccgaagtatctatcgaagaaattgattccgatgactcggagcactacacaaccgcatcggagaatggcgagtacggtgtagattggtttaatgcaaatacatttgccaatatatcatacgattgtagagatgtacattatatgacaggggccaattttgaatacattaaaaatcaagaaaacaaaaaaaaaaaaaaaaaaaaaaaa

SEQ ID NO: 11 - DNA, 2065 bp, Ceratitis capitata
  5′UTR (1)..(75)
  3′UTR (1774)..(2065)
attcattgtcacataagtgttactctaggtcagactacaaagtgttggaattgtgaaaattaaaaaaaaaaaaaaatggagcaagtgctgtctaataaaaatgacaagtcgtctaacggaaatggcgtcttagtgcaatgtatacaagtgacatataaaaaaacaccggtgtattctctacgtcaatattttcgacaatttggcgccttccgaaatttcgaaacagcatatgaccttaacccacaacaacttgaaccgatcaagagactccttataacatacgttgaacaagaggcagcccgccgagttttgcaaaacgaatctcattttgtggaaaacgagattgttaatgttaaaagtacaaatagtcggcaagaactcgttaaggaaaatgtttgtgaaacagagcgtgaaaatttggaaatcgctcacactagggaaaaagccttcaacattttacaattgcctgaattttgcattcatgctatactcgaacgtatggcaccaggtgatcaaatacgttttgtgcagtcgcacagaagttttcaaacgatctttgaaacgttttctcgacgcaaatataaaaagttcatatggaataaaacacaaagtatgactatgggtgaaatacgtgatttcttgagcatagcaggcaaatatatggtggaattgaatggtgaaatacgtaatagatattatgatgacgtaatgaatctattaatcgtttactgtccgaatttcgaaaaagttacgctctcagacatatacattaatccagatagttttcggaattttttgagaggactgccaaatttacgggaattgaatttaacgtattatcaattagatgagagggcatttgcagcactgagggacgctaaaaaactgcgtgtgctgaaattgtccaatatgttccacataagaggcaaacatcttcaaaccctgacgcaactggagcatctctctctgcaccactgttggttggttcgaagtaaacatttaattaaactctgtgaggcgttggtaaacttacgcacactggacatacgcggcatacactacgcatcaccctgtttttttcaagtaatcgacacacattgcaagcgactcgaggtgttgaagatgtgcgcaccggaatttaattacgatcgtgtggcattattgccacgcctcaaacatctcgaaatcaaatatcccacacccgttgataggttgacaataatctcacatttggctcaacacaaggcggatcaactcgaagtactgaaaatatacaccagaaaatgtctaaccgacaaagacgtcacaagtttatcagaattacgtgagttgagagaactttccatcgcttttaatcgggataccgtgaccaatggcgctttagcgaaattatgtaaattgaagcagctggaagagttgacaatcgccgcttgcgacgaggtgaccgatgatggactgttgaagttggtaaaaaattgtgagaaactgagcactctaaatatacaatattgtaatagaattacaaatgattttgtgacggacgtcgcttcattggcacgacatcgttatagaaaattaacaatttacacgtttggcacggcggtggacaagaaaaagttaatacagcttccaaagtataaggaggcggctgagcgttttggtttaaatattgagtttgagttttcaaaaacaggattatcggattatgaatatgtttttacactttttgaaaatgagttgcttgaacgtcgactcattgcaagagggttatagaagcagatacgtgtatatacatatgtttgtatgtaaaaactaaaaaaaaaaaatattaaatttcttttgggtggaatatataattagaattgtacaacactattcttttatgtatacatatatatttttaataaagggtcattcattagcgtctgacaactcaaactgccacttcaaacgcgcccttaatccgattgcccaatttttaatgccacattttatcattgtgaaacaaacgtttaaaataaataaaatttactaccgaaattccttacttaaaaaaaaaaaaaaa

SEQ ID NO: 12 - DNA, 820 bp, Ceratitis capitata
ggccaagtctggactgtaaggttggtgattaaaaatcgctcttatattgtcaacttatagaaacatatttttctttatgatcaatcaataatattctttccgagttccaaacgaccgtttgccatgagtttaccagctgactgtgaaatcttaaaccgaaaattgcactcatagactttaaaaatgtttgtatcggatacaccaagcgcttctgtaagttgtctcttctttggtggacgtcgttcggggacaatttttttctaccttttggacaaattctcgcgtgatcaaaataaagagcaataaaaaactctctgctctactcacgttcgattagtgtttcaagtctttggagaagagtgatggcagaaaggtacttgtcgagctggagagtatagttttgtcatctacaacggcatccaggtgttgatagagtgcctttttccattccaaacgttctggtatgcattcctgtatgaagatggcacctttcaccaactcattaatatcagttgtgcccgaaatgagcgcgaattgttggttagcactgagtttgccacgcagtaagcccttcgattccaagcttttcagctccttttctgtctcacgtaaggcattttcgatttgtgttggtacactgtcgtagagcactacttgatagcccaccgaggcgaagagcatggaccatgaacgtccgataagaccgctaaatgttaaatattagtataaattttgtatttttttattgacgtttcacttgatatttttcgttttctttgaaagtcacctgcccacaataccaactttgcattgactggcc

SEQ ID NO: 13 - DNA, 565 bp, Ceratitis capitata
ccggatatttaagtgataatcttttaaaccgtatctaaatacatattgtataaatataaataaattttgttactaatttatttattaatgttcattttctccgttaaaaaatctctaattacagttcttgcacgcctacgtttgatacttcctttgtaaacgtgtgactaataaaattcgccatcacgacgcagtacgcacttttaaccgttaaacacattatttgtgtctacaaggtgaagaaaaaattcatcgaatcagttaataatacataaaatactcgctaatcgccgacattcagcagatattggtggaacaaacgcttgccaacgatcggacactagtcacagccgcaaccgcatacgccgtgtagtaacgtccattctatcaaaccaacgtcgctttagccaaaccaacacccacccacacacacacactcacaatgcccgttcaatctcccatcgcgattagcaatcgtgccattgaacatatcgatgatgtcgatcccttgaaatatcatggccattgggaaccagttggtgttgcacgcgtacaaaatatccgg

SEQ ID NO: 14 - DNA, 43 bp, Drosophila melanogaster
aggagcgccggagtataaatagaggcgcttcgtctacggagcg

SEQ ID NO: 15 - DNA, 189 bp, Drosophila melanogaster
cacgtttgcttgttgagaggaaaggttgtgtgcggacgaatttttttttgaaaaccggtgatagagcctgaaccagaaaagataaaagaaggctataccagtgggagtacacaaacagagtaagtttgaatagtaaaaaaaatcatttatgtaaacaataacgtgactgtgcgttaggtcctgttcatt

SEQ ID NO: 16 - DNA, 1008 bp, Artificial
  tTA gene
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 17 - DNA, 52 bp, Artificial
  attP site
gtgccccaactggggtaacctttgagttctctcagttgggggcgtagggtcg

SEQ ID NO: 18 - DNA, 29 bp, Ceratitis capitata
ggttttcggattctttgcaattcacgatg

SEQ ID NO: 19 - DNA, 22 bp, Ceratitis capitata
cgctctcggaaagatcagtacg

SEQ ID NO: 20 - DNA, 23 bp, Ceratitis capitata
ccacagccacccgacgccattgg

SEQ ID NO: 21 - DNA, 28 bp, Ceratitis capitata
cttcgagtgctccgttgaagatgatagc

SEQ ID NO: 22 - DNA, 20 bp, Ceratitis capitata
cggaaggcgccaaattgtcg

SEQ ID NO: 23 - DNA, 26 bp, Ceratitis capitata
gcgagaggttgttgtatgtccggcac

SEQ ID NO: 24 - DNA, 26 bp, Ceratitis capitata
catacgttgaacaagaggcagcccgc

SEQ ID NO: 25 - DNA, 24 bp, Ceratitis capitata
gtgtaattgctggtcggtcgacac

SEQ ID NO: 26 - DNA, 26 bp, Artificial
  corresponds to pTetOff
catagaagacaccgggaccgatccag

SEQ ID NO: 27 - DNA, 27 bp, Artificial
  corresponds to pTetOff
gcatgcggagatctaagcttggtcgag

SEQ ID NO: 28 - DNA, 29 bp, Ceratitis capitata
gtccgtagagatctgccgcagcattgtcc

SEQ ID NO: 29 - DNA, 29 bp, Ceratitis capitata
agcattctagacatactggccggcggagc

SEQ ID NO: 30 - DNA, 24 bp, Artificial
  Primer mfs-117
gatcggccggccttggcgcgccta

SEQ ID NO: 31 - DNA, 24 bp, Artificial
  Primer mfs-118
gatctaggcgcgccaaggccggcc

SEQ ID NO: 32 - DNA, 29 bp, Ceratitis capitata
gtccgtgcgaattctgtatgcataagtcg

SEQ ID NO: 33 - DNA, 28 bp, Ceratitis capitata
cgtaggagctctagacatttctgtgatg

SEQ ID NO: 34 - DNA, 27 bp, Ceratitis capitata
tccttattgaccgtacgaccttgtggc

SEQ ID NO: 35 - DNA, 35 bp, Ceratitis capitata
gtccgtgcgaattctaaattctgaaagctatctgg

SEQ ID NO: 36 - DNA, 43 bp, Ceratitis capitata
tcaggtctctctagacatttttttttttaattttcacaattct

SEQ ID NO: 37 - DNA, 26 bp, Ceratitis capitata
cacaggagctgaagtgccaactgatg

SEQ ID NO: 38 - DNA, 29 bp, Ceratitis capitata
cttctgtggttacttttatgagttcgccg

SEQ ID NO: 39 - DNA, 20 bp, Ceratitis capitata
tccgtccgccgtcatattgg

SEQ ID NO: 40 - DNA, 23 bp, Ceratitis capitata
tagccagattccgtttcacattc

SEQ ID NO: 41 - DNA, 26 bp, Ceratitis capitata
cttcgagttcgatcactgcacaattc

SEQ ID NO: 42 - DNA, 22 bp, Ceratitis capitata
agttttcagccgcaggctttcg

SEQ ID NO: 43 - DNA, 24 bp, Ceratitis capitata
cgcataaactggatgtggcacgcc

SEQ ID NO: 44 - DNA, 27 bp, Ceratitis capitata
acattgaaatgtagtgaaatggtggcg

SEQ ID NO: 45 - DNA, 41 bp, Ceratitis capitata
agcattctagacatattggattttcaataaacaagtatttc

SEQ ID NO: 46 - DNA, 37 bp, Ceratitis capitata
acagtccatggctatccgtgctttcgctacatttatc

SEQ ID NO: 47 - DNA, 39 bp, Ceratitis capitata
acagtccatggcagtggtcactcatcatccttcaaaatg

SEQ ID NO: 48 - DNA, 46 bp, Ceratitis capitata
tcaggtctctctagacattttgataattgaacactttaccacgctc

SEQ ID NO: 49 - DNA, 56 bp, Artificial
  attP primer
aattcgtgccccaactggggtaacctttgagttctctcagttgggggcgtagggtc

SEQ ID NO: 50 - DNA, 56 bp, Artificial
  attP primer
aattgaccctacgcccccaactgagagaactcaaaggttaccccagttggggcacg

SEQ ID NO: 51 - DNA, 57 bp, Artificial
  attP primer
catgggtgccccaactggggtaacctttgagttctctcagttgggggcgtagggtcg

SEQ ID NO: 52 - DNA, 57 bp, Artificial
  attP primer
catgcgaccctacgcccccaactgagagaactcaaaggttaccccagttggggcacc

SEQ ID NO: 53 - DNA, 57 bp, Drosophila melanogaster
ctagggtgccccaactggggtaacctttgagttctctcagttgggggcgtagggtcg

SEQ ID NO: 54 - DNA, 57 bp, Drosophila melanogaster
ctagcgaccctacgcccccaactgagagaactcaaaggttaccccagttggggcacc

SEQ ID NO: 55 - DNA, 20 bp, Artificial
  DSRed forward primer
ccaccacctgttcctgtagc

SEQ ID NO: 56 - DNA, 21 bp, Artificial
  DSRed reverse primer
ttgcctttcgccttattttag

SEQ ID NO: 57 - DNA, 20 bp, Artificial
  Primer mfs-333
gctcctccaagaacgtcatc

SEQ ID NO: 58 - DNA, 20 bp, Artificial
  Primer mfs-334
tggtgtagtcctcgttgtgg

SEQ ID NO: 59 - DNA, 40 bp, Artificial
  Primer mfs-335
gtaatacgactcactatagggcggcggtcacgaactccag

SEQ ID NO: 60 - DNA, 18 bp, Artificial
  Primer mfs-336
gtgagcaagggcgaggag

SEQ ID NO: 61 - DNA, 35 bp, Artificial
  Primer mfs-41
gcggataacaatttcacacaggaaacagctatgac

SEQ ID NO: 62 - DNA, 31 bp, Artificial
  Primer mfs-42
cccagtcacgacgttgtaaaacgacggccag

(19) United States
(12) Patent Application Publication
(10) Pub. No.: US 2011/0088105 A1
(43) Pub. Date: Apr. 14, 2011

(54) DEVELOPMENT STAGE-SPECIFIC LETHALITY SYSTEM FOR INSECT POPULATION CONTROL

(76) Inventors: Ernst A. Wimmer, Gottingen (DE); Marc F. Schetelig, Gottingen (DE)

(21) Appl. No.: 12/933,320

(22) PCT Filed: Mar. 19, 2009
(86) PCT No.: PCT/EP2009/053240

§ 371 Date: Dec. 10, 2010

Related U.S. Application Data
(60) Provisional application No. 61/038,210, filed on Mar. 20, 2008.

(51) Int. Cl. A01K 67/033; C12N 15/85

(52) U.S. Cl. 800/13; 435/320.1; 800/21

(57) ABSTRACT

The application describes a transgenic insect comprising a developmental stage-specific lethality system. The developmental stage-specific lethality system comprises a first gene expression cassette comprising a first promoter/enhancer element of a developmental stage-specific gene derived from an insect pest species, preferably from a member of the family Tephritidae, a first component of a transactivating system, a second gene expression cassette comprising a second component of the transactivating system, a second promoter responsive to the activity of the transactivating system, and a lethality inducing system. Also, the application describes a method of controlling reproduction in an insect population of interest, comprising providing a plurality of insects according to the invention and allowing the insects to interbreed with insects of the population of interest. Further, the application describes a method for producing transgenic insects comprising a developmental stage-specific lethality system comprising providing a set of insects comprising gene expression cassettes according to the invention, and further evaluating the insects or offspring thereof for functionality of the developmental stage-specific lethality system. Also, the application describes the use of a transgenic insect according to the invention for controlling reproduction in an insect population of interest. Further, the application describes a developmental stage-specific lethality system for use in a transgenic insect comprising gene expression cassettes according to the invention.

### FIELD OF THE INVENTION

The present invention relates to transgenic insects that are useful in biological methods for controlling pest insects such as the sterile insect technique (SIT). More specifically, the invention relates to transgenic insects comprising a developmental stage-specific lethality system, methods for producing such insects, and methods of their use in controlling reproduction in an insect population of interest. Furthermore, the invention provides a developmental stage-specific lethality system for use in insects based on developmental stage-specific lethal transgene combinations derived from insect pest species, particularly from members of the family Tephritidae.

### BACKGROUND OF THE INVENTION

Many insects heavily damage crops, fruit, and forests or transmit diseases to animals and humans. Current control efforts mostly rely on the use of insecticides, but these chemicals can have adverse side effects, and costs for developing new chemical products to overcome e.g. insecticide resistance are increasing.

In contrast, biological methods such as the sterile insect technique (SIT) are environmentally friendly and very effective in species-specific control of pest insects. Generally, the SIT reduces a pest population by mass release of reproductively sterile male insects into a wild type (WT) population of the same species. This leads to the decrease of progeny by competition of sterilized males with WT males for WT females. Ultimately, if enough males are released for a sufficient amount of time, a total eradication of the pest population can be achieved. In SIT programs, besides the monitoring, mass rearing, and release of the pest species, the sterilization procedure is of major importance. Because of its species-specificity, SIT is considered an ecologically safe procedure and has been successfully used in area-wide approaches to suppress or eradicate in entire regions pest insects such as the pink bollworm Pectinophora gossypiella in California, the New World screwworm fly Cochliomyia hominivorax in North and Central America, and various tephritid fruit fly species in different parts of several continents.

Typically, in the current SIT approaches, the males are sterilized by radiation, which has the disadvantage that sterility and competitiveness of the insects are indirectly correlated. Therefore, in some programs lower doses of radiation are used to generate sterile insects, which show increased fitness and are more competitive, but are mostly only partially sterile. However, in preventional release programs in areas that are still pest-free, it is crucial to release only completely sterile flies in order to avoid an establishment of the pest or to control the problem of a re-infestation in eradicated areas. Thus, such programs have to use 100% sterile insects if a novel introduction of insect pests is to be avoided. However, due to the high dose of radiation required for complete sterility of conventionally sterilized insects, the competitiveness of such insects is generally reduced.

Among the about 250 known insect pest species of the Tephritidae family, the Mediterranean fruit fly (medfly), Ceratitis capitata (Wiedemann; Diptera: Tephritidae), is one of the most devastating and economically important ones.

In THOMAS (2000), a transgenic system for achieving female-specific lethality in Drosophila melanogaster is introduced, based on a tetracycline-repressible transactivating system controlling the expression of lethal genes. Nevertheless, this system has not been transferred to pest insects like Ceratitis so far. In addition, this system only results in a killing of females, and female-specific lethality occurs in late developmental stages like late larval stages or pupae. This system is also described in WO 01/39599 A2.

GONG (2005) describe a dominant lethal genetic system for medfly based on overexpression of the tetracycline-repressible transcription factor tTA. In the presence of tetracycline, tTA expression is repressed, whereas in the absence of tetracycline, tTA levels increase by an autoregulatory loop mechanism to lethal levels. However, the article reports that the system still allowed the development of a significant proportion of larvae, pupae, and adults, which is a downside regarding any actual use in insect-infested agricultural areas. This system is also described in WO 2005/012534.

In FU (2007), a female-specific lethality system designed for use in the insect pest medfly is described. The system relies on sex-specific alternative splicing of a dominant lethal transgene. By way of insertion of a female-specific intron into the gene coding for the tetracycline-repressible transcription factor tTA, repressible dominant lethality specific for female medflies could be achieved. But this female-specific lethality occurs predominantly in pupae, which would increase the diet consumption by unwanted females during mass rearing compared to the female-specific embryonic lethal sexing system based on the Y-linked rescue of a tsl mutation, which is currently used (FRANZ (2005)). In addition the lethality is limited to females. The system is also described in WO 2007/091099.

In HORN AND WIMMER (2003), a first approach to cause reproductive sterility by transgene-based embryonic lethality without the need of radiation is described for the non-pest insect Drosophila melanogaster. The system of HORN AND WIMMER (2003) is based on the transmission of a transgene combination that causes embryo-specific lethality in the progeny. To limit the effect of the transgenes to the embryonic stage, promoter/enhancers (P/Es) from cellularization-specifically expressed Drosophila melanogaster genes D.m. serendipity α and D.m. nullo were chosen to drive the expression of the tetracycline-controlled transactivator (tTA). The expressed transactivator then activates the expression of the lethal effector gene hid^{Ala5}, which itself was placed under control of the D. melanogaster P basal promoter. The authors report that other promoters such as the cytomegalovirus core promoter or the minimal promoter of the heat-shock gene hsp70 did not yield functional transgenic fly lines. Finally, effective expression of the lethal effector gene hid^{Ala5} resulted in embryonic lethality in some of the resulting fly lines.

SCHETELIG (2007) report an attempt to transfer the sterility system of HORN AND WIMMER (2003) from Drosophila melanogaster directly to the medfly Ceratitis capitata. However, later results show that the system proved to be not functional in medfly (M. F. Schetelig, A. M. Handler, E. A. Wimmer, unpublished results). The paper further describes the outlines for a search for cellularization-specific genes in medfly.

Thus, there is a need in the art for an improved biological method for controlling insect pest populations that overcomes the problems currently associated with the SIT based on irradiation of male insects.

### SUMMARY OF THE INVENTION

Accordingly, the present invention relates to a transgenic insect comprising a developmental stage-specific lethality system comprising a first gene expression cassette comprising a first promoter/enhancer element of a developmental stage-specific gene derived from an insect pest species, preferably from a member of the family Tephritidae, a first component of a transactivating system, a second gene expression cassette comprising a second component of the transactivating system, a second promoter responsive to the activity of the transactivating system, and a lethality inducing system, as defined in the claims. Also, the invention relates to a method of controlling reproduction in an insect population of interest, comprising providing a plurality of insects according to the invention and allowing the insects to interbreed with insects of the population of interest, as defined in the claims. Further, the invention relates to a method for producing transgenic insects comprising a developmental stage-specific lethality system comprising providing a set of insects comprising gene expression cassettes according to the invention, and further evaluating the insects or offspring thereof for functionality of the developmental stage-specific lethality system, as defined in the claims. Also, the invention relates to the use of a transgenic insect according to the invention for controlling reproduction in an insect population of interest, as defined in the claims. Further, the invention provides a developmental stage-specific lethality system for use in a transgenic insect comprising gene expression cassettes according to the invention, as defined in the claims.

### DETAILED DESCRIPTION OF THE INVENTION

The present invention shows that, unexpectedly, a developmental stage-specific lethality system could be successfully provided in insects based on developmental stage-specific lethal transgene combinations derived from insect pest species, particularly from members of the family Tephritidae. The inventors could show that when transgenic insects from lines according to the invention are mated to corresponding wildtype insects, most or all progeny die during early development. The observed complete or near complete lethality of the insect progeny after mating of transgenic individuals with wildtype individuals, could allow a release of transgenic insects into areas of interest without the need of sterilization by way of radiation. Moreover, insects according to the invention proved highly competitive in laboratory and field cage tests, and therefore may be used immediately for evaluation in mass rearing tests. Thus, the present invention offers a means to overcome the disadvantage of sterilizing insects by way of radiation that is currently employed in pest management programs. Further, the use of transgenic insects according to the invention displaying complete or near complete lethality in early developmental stages, offers the further advantage of avoiding a hatching of progeny in areas where the insects are released, thus avoiding fruit or crop damage caused by the larvae. Even more importantly, by preventing a hatching of progeny, the present invention also provides means to avoid the ingression of transgenes into the wild insect population. In addition, an accidental escape of Ceratitis from mass-rearing facilities would currently cause problems, if the insects have not been sterilized before. However, by using the embryonic lethal lines, the escaped insects would be 100% reproductively sterile. Thus they would not cause any problems even when escaped into preventional area. In this direction, transgenic insects can increase the safety of the mass-rearing process for operational SIT programs. All this makes the described insects suitable for use even in preventional release programs, where sterile insects are released in pest-free areas to prevent pest reinfestations, and where 100% sterility is a prerequisite. Thus, the system may prove to be a promising tool for conferring sterility to insect populations, preferably pest species, and may provide great advantages in environmentally friendly pest control techniques like the sterile insect technique (SIT) against insect pests occurring in economically important areas, such as farmland and orchards. Finally, a combination of the new developmental stage-specific lethality system according to the invention with the genetic background of well-established organisms suitable for genetic sexing, such as medfly tsl-lines, could become a powerful tool to improve current SIT programs.

Thus, in a first aspect, the present invention relates to a transgenic insect comprising a developmental stage-specific lethality system comprising a) a first gene expression cassette comprising, in operative linkage, (i) a first promoter/enhancer element of a developmental stage-specific gene derived from an insect pest species, or a functional derivative of said promoter/enhancer element, (ii) a first component of a transactivating system, whose activity is controllable by a suitable exogenous factor, and b) a second gene expression cassette comprising, in operative linkage, (i) a second component of the transactivating system, (ii) a second promoter that is responsive to the activity of the transactivating system, and (iii) a lethality inducing component. Preferably, the first promoter/enhancer element or a functional derivative of said promoter/enhancer element is derived from a member of the family Tephritidae.

An insect according to the invention is an animal belonging to the class insecta, preferably to the order Diptera, further preferably to the suborder Brachycera, further preferably to the family Tephritidae, more preferably to the genus Ceratitis, even more preferred to the subgenus Ceratitis, and most preferably to the species Ceratitis capitata. There are various C. capitata wild strains e.g. from Egypt (strain EgII), Argentina (strain Arg), Costa Rica, Hawaii, or Portugal, which are each known to have specialized courtship behavior and can be distinguished from each other. It is preferred that the insect according to the invention belongs to an insect pest species. Agricultural insect pests, for example, inflict damage on agricultural products such as fruits, crops, vegetables, farm animals, and are therefore of economical relevance. Other pest insects are insect disease vectors e.g. mosquitoes, which transmit human and animal diseases like malaria, dengue or yellow fever, and are therefore of medical relevance.

Generally, the term “insect pest species” includes injurious or unwanted insects and insects recognized as a destroyer of economic goods or a risk for animal and human health, e.g. by carrying germs within human habitats. Insect species often become pest species when the ecological balance is interrupted by human intervention or natural events, which leads to an overgrowth of these species. However, it is also contemplated that the developmental stage-specific lethality system of the invention can be used in insect species that are not pest insects.

The term “developmental stage-specific” as used herein refers to a system or a gene that is active or capable of being activated during a certain stage during development or adult life of the animal. Preferably, the term developmental stage-specific as used herein refers to early stages during development of the organism. Thus, the system according to the invention is activated during development of the transgenic insect, and preferably causes lethality already in embryos. In further scenarios, lethality would occur primarily in larval, pupal, or adult stages, even though larvae would then develop and increase the damage in comparison to lethality occurring already in embryonic stages. Thus, embryonic stages are preferred.

A person skilled in the art will know how to identify specific stages during development or adult life of an animal in question. In a preferred embodiment, a developmental stage-specific system or gene is a cellularization-specific system or gene, i.e. is a system or gene active or capable of being activated during cellularization. Typical characteristics of the cellularization stage are known to the skilled person. For example, in insects, the cellularization is the synchronous introgression of membrane furrows to separate single blastoderm nuclei. This process can be divided into slow and fast phase reflecting the rate of membrane invagination. The process of cellularization involves integrating mechanisms of cell polarity, cell-cell adhesion and a specialized from of cytokinesis, which ends up in a monolayer of blastoderm cells.

Examples for developmental stage-specific genes are the genes C.c.-serendipity α (SEQ ID NO. 7), C.c.-CG2186 (SEQ ID NO. 8), C.c.-slow as molasses (SEQ ID NO. 9), C.c.-sub2_{—}99 (SEQ ID NO. 10), C.c.-sub2_{—}63 (SEQ ID NO. 11), or C.c.-sub2_{—}65 (SEQ ID NO. 6), as described herein. These genes are active during cellularization, whereas C.c.-sub2_{—}63 is, in addition, expressed during germ band elongation (FIG. 1).

The use of a system or a gene according to the invention that is active or capable of being activated during the developmental stages, particularly early developmental stages, of an insect offers various advantages. Firstly, released males carrying the system and mating to wildtype females offer the advantage of inhibiting larval development in the field, which ensures crop quality and quantity. Second, the described promoters from developmental stage-specific genes are supposed to be activated early, but also exclusively in embryos. Other promoters, which are active in early but also in later stages, might cause side effects leading to a decreased fitness of the strains and a lowered efficiency during field releases. Third, using a lethality system that is active during early developmental stages of transgenic insects has the additional advantage that an ingression of transgenes into the wild insect population may be avoided after the intentional or unintentional release of transgenic insects.

As used herein, the term “in operative linkage” refers to the positioning of an element in the gene expression cassette according to the invention, or to the positioning of a nucleic acid, in such a way as to permit or facilitate transcription and/or translation of the nucleic acid in question. In the context of the invention, the term “in operative linkage” refers to any order of arrangement of the elements or components of a gene expression cassette according to the invention permitting functional interactions of the elements or the component in question. For example, “in operative linkage” can mean that a set of DNA sequences are contiguously linked, or that enhancer elements are placed in a position so as to exert regulatory effects onto corresponding genes.

A “promoter/enhancer element” as used herein is typically a DNA sequence located 5′ to a DNA sequence to be transcribed, and is typically positioned upstream of the ATG of the first exon of a coding sequence or a transcription start side. Generally, a promoter/enhancer element as used herein refers to a combination of a promoter region, e.g. the region upstream of a coding region to which RNA polymerase binds, and a cis-regulatory sequence that can increase transcription from an adjacent promoter.

In a preferred embodiment, the promoter/enhancer element according to the invention is the promoter/enhancer element of a developmental stage-specific gene derived from a member of the family Tephritidae. Preferably, the gene is derived from a member of the class insecta, preferably of the order Diptera, further preferably of the suborder Brachycera, further preferably of the family Tephritidae, more preferably of the genus Ceratitis, even more preferred of the subgenus Ceratitis, and most preferably of the species Ceratitis capitata, or any specialized Ceratitis capitata strain as described above. In a further preferred embodiment, the promoter/enhancer element of the invention is selected from the group consisting of the promoter/enhancer element of the C.c.-serendipity α gene (SEQ ID NO. 1), the promoter/enhancer element of the C.c.-CG2186 gene (SEQ ID NO. 2), the promoter/enhancer element of the C.c.-slow as molasses gene (SEQ ID NO. 3), the promoter/enhancer element of the C.c.-sub2_{—}99 gene (SEQ ID NO. 4), the promoter/enhancer element of the C.c.-sub2_{—}63 gene (SEQ ID NO. 5), and the promoter/enhancer element of the C.c.-sub2_{—}65 gene, which gene has SEQ ID NO. 6. Functional derivatives of these promoter/enhancer elements are included, and are further described below. Most preferably, the promoter/enhancer element is the promoter/enhancer element of the C.c.-serendipity α gene (SEQ ID NO. 1), or a functional derivative thereof.

A promoter/enhancer element of a developmental stage-specific gene can be derived from any organism of interest by various techniques known in the art. For example, i) if the sequenced genome of the organism is available, specific primers can be designed for isolating the desired promoter/enhancers; ii) if a fragment of the gene is known, but no sequenced genome of the organism is available, RACE (rapid amplification of cDNA ends) and/or inverse PCR can be used to isolate flanking regions of the gene fragment, which include the promoter/enhancer elements; iii) if the genome of an organism is not sequenced and also no fragment of the desired gene is known from the organism of interest, degenerate primers can be created (based on protein alignments of known homologous genes from other organisms) and used in PCR reactions using an embryonic cDNA pool of the organism of interest, as described in SCHETELIG (2007). In light of the present disclosure this method allows to isolate conserved parts of the gene, and in a second step to isolate the flanking regions as described in ii). In method iv), if degenerative primer PCRs are not successful in isolating stage-specific expressed genes e.g. because of low conservation of the endogenous gene to the known homologs, a differential display can be used to isolate genes, which are differentially expressed between or among different cells, tissues or developmental stages such as the cellularization stage. With this method, also parts of developmental stage specific genes can be isolated and in a second step, the promoter/enhancers can be isolated as described in ii). Method (v): another example for isolating promoter/enhancer elements is an enhancer-trap approach. Such a system can base on a controlled mobilization of a broad-range transposable element e.g. piggyBac (HORN (2003b). A jumpstarter element expressing the respective transposase (e.g. piggyBac transposase) gene is used to mobilize a non-autonomous mutator element based on the respective transposable elements. This mutator element carries a heterologous transactivator gene that serves as a primary reporter of enhancer activities. The heterologous transactivator than activates a secondary reporter within a responder element, which is used for the visible detection of the enhancer activity.

Generally, the term “promoter/enhancer element” according to the invention is meant to include functional derivatives of the promoter/enhancer elements of the invention. A “functional derivative” of a promoter/enhancer element according to the invention or of any other nucleic acid sequence of the invention is derived from the original, i.e. wildtype, nucleic acid sequence in question, an artificially modified version of the original sequence or a naturally occurring allele of the original sequence. Preferably, a functional derivative of a promoter/enhancer element has e.g. 50%, 55%, 60%, 65%, 70%, 75%, 80%, 85%, 90%, 95%, or 100% nucleic acid sequence identity to the original or wildtype nucleic acid sequence over a length of at least 15 contiguous nucleotides, when the best matching sequences of both nucleic acid sequences are aligned. Preferably, a functional derivative of a promoter/enhancer element according to the invention has 50%, 55%, 60%, 65%, 70%, 75%, 80%, 85%, 90%, 95%, or 100% nucleic acid sequence identity over a length of at least 15 contiguous nucleotides to any of the nucleic acid sequences selected from the group consisting of SEQ ID NOs. 1, 2, 3, 4, and 5. Generally, a nucleic acid molecule has “at least x % identity” over a defined length of nucleotides with another nucleic acid sequence or any of the SEQ ID NOs. shown above if, when a sequence of 15 or more contiguous nucleotides of the nucleic acid sequence in question is aligned with the best matching sequence of the other nucleic acid sequence or any of SEQ ID NO. 1-5, the sequence identity between those to aligned sequences is at least x %. Such an alignment can be performed using for example publicly available computer homology programs such as the “BLAST” program provided at the NCBI homepage at http://www.ncbi.nlm.nih.gov/blast/blast.cgi, using the default settings provided therein. Further methods of calculating sequence identity percentages of sets of nucleic acid sequences are known in the art. The term “functional derivative” is also meant to include truncated or otherwise altered versions of a promoter/enhancer element in question, as long as the functionality of the derivative is maintained. Also, homologous sequences from other species are included, when their function is conserved between species.

Further, the term “functional derivative” of a promoter/enhancer element according to the invention requires that the derivative of a promoter/enhancer element in question is “functional”, i.e. shows the biological activity of the unchanged, i.e. wild type promoter/enhancer element. The biological activity shown by a derivative can be the full activity when compared to the wildtype sequence under identical conditions, or can be less than full activity, e.g. 10%, 20%, 30%, 40%, 50%, 55%, 60%, 65%, 70%, 75%, 80%, 85%, 90%, 95%, or 99% of the activity of the wildtype sequence when compared under identical conditions. The biological activity of a promoter/enhancer element can be evaluated by a skilled person e.g. by comparing the expression levels of a gene under control of either a promoter/enhancer element derivative or the corresponding wildtype promoter/enhancer element.

The lethality system according to the invention also comprises a first and a second component of a transactivating system, wherein the activity of the transactivating system is controllable by a suitable exogenous factor. A transactivating system is suitable for use within the invention if it is capable of serving as a mediator between the activity of the first promoter/enhancer element of the first gene expression cassette according to the invention and the second promoter and the lethality inducing system of the second gene expression cassette according to the invention. Further, it is preferred that the transactivating system is controllable by a suitable exogenous factor. In a preferred embodiment, the activity of the transactivating system can be repressed in the presence of the exogenous factor. For example, a repression of the activity of the transactivating system can be measured by measuring the level of lethality caused by the lethality system of the invention in the presence and without the presence of the exogenous factor. Preferred transactivating systems in the context of the invention are, for example, the Tet-Off or the Tet-On system as described in MCGUIRE (2004).

In a preferred embodiment, the transactivating system is the Tet-Off system. Generally, the first component of the Tet-Off transactivating system is capable of expressing the tetracycline-repressible transactivator (tTA) (SEQ ID NO. 16) or a functional derivative thereof, and wherein the second component of the TET-OFF system comprises a tTA-responsive element. Typically, the suitable exogenous factor is tetracycline or a functional derivative or functional analog thereof. Examples for functional derivatives and functional analogs of tetracycline include, but are not limited to doxycycline, 4-epidoxycycline, anhydrotetracycline, 4-epi-oxytetracycline, chlorotetracycline, and cyanotetracycline. A skilled person can determine suitable amounts of exogenous factor for use in accordance with the invention e.g. by the methods described in the exemplifying section herein. Typically, if the TET-OFF system is used, tetracycline is supplied in concentrations ranging between 1 and 100 μg/ml.

Further examples for suitable transactivating systems are known in the art and include e.g. the GAL4-ER system, which is based on steroid hormone responsive transcription factors, or the classical EAL4-UAS system (TARGET), which is based on the GAL4 transcriptional activator from yeast as a first component, and UAS binding sites together with a temperature-sensitive allele of the GAL80 factor as a second component, as described for example in MCGUIRE (2004). Suitable exogenous factors for controlling these systems are e.g. steroid hormones or physical effects like applying a heat shock.

The second gene expression cassette according to the invention comprises a second promoter that is responsive to the activity of the transactivating system. Preferably, the second promoter has no regulatory effect such as gene transcription in the absence of activity of the transactivating system, and only becomes active when the transactivating system is activated. Preferably, the second promoter is operatively linked to the lethality inducing component of the lethality system, and upon activation drives the activity or expression of the lethality inducing component. In a preferred embodiment, the second promoter is selected from the group consisting of the Drosophila melanogaster hsp70 basal promoter (SEQ ID NO: 14) and the Drosophila melanogaster P basal promoter (SEQ ID NO: 15), or a functional derivative of any of these promoters as defined above. Preferably, the second promoter is the Drosophila melanogaster hsp70 basal promoter (SEQ ID NO: 14) or a functional derivative thereof as defined above. A “basal promoter” is typically a promoter sequence that is sufficient to promote gene expression in the presence of transcription factors. A basal promoter is not able to start transcription without additional transcription factors.

A “lethality inducing component” in accordance with the present invention is a component capable of causing lethality in a cell or an organism carrying the second gene expression cassette of the invention. “Lethality” of a lethality system as used herein can be expressed as “% lethality” by determining the percentage of cells or organisms that die after activation of a lethality system. Preferably, the lethality-inducing component of the invention causes 50%, 55%, 60%, 65%, 70%, 75%, 80%, 85%, 90%, 95%, or 100% lethality upon activation for a suitable amount of time. 100% lethality is referred to as “complete lethality” and is preferred. In a further preferred embodiment, the lethality inducing component of the invention is under control of the second promoter of the second gene expression cassette, and its activity is in turn controllable by activation or deactivation of the controllable transactivating system. A variety of lethality inducing components can be used in accordance with the present invention. Preferably, the lethality-inducing component is selected from the group consisting of a pro-apoptotic gene, an apoptotic gene, toxins, hyperactive cell-signalling molecules and the method of systemic RNA interference (RNAi) to genes, which are important during development. Examples for pro-apoptotic genes include, but are not limited to head involution defective (hid), and preferred examples are the phosphoacceptor-site mutant versions of hid, and most preferred the mutant version hid^{ala5} (BERGMANN (1998)), or a functional derivative of this gene as defined under “functional derivative” of nucleic acid sequences above. Examples for apoptotic genes include, but are not limited to hid, grim and reaper. Examples for toxins include, but are not limited to ricin, Diphteria toxins, and shiga toxins. Examples for hyperactive cell-signalling molecules include, but are not limited to genes involved in oncogenesis like ras. Examples for target gene transcripts of specific dsRNA-induced lethality are early embryonic active gene transcripts, and preferred examples are the transcripts of the target genes serendipity α and slow as molasses.

In a further preferred embodiment, the first and the second gene expression cassette of the invention, or the first or the second gene expression cassette, further comprise(s) a minimal attachment P (attP) site (SEQ ID NO: 17), or a functional derivative thereof, as defined under “functional derivative” of other nucleic acid sequences above. Minimal attP sites are described e.g. in GROTH (2004), and offer the advantage of site-specific integration at an attP site, which allows a modification of the transgene contained therein.

In another preferred embodiment, the first and the second, or the first or the second gene expression cassette according to the invention further comprise(s) one or more marker genes that allow detection when expressed in an insect of the invention. A variety of suitable marker genes are known in the art offering expression detection by means such as optical or immunological methods. Preferably, the marker genes used in the context of the invention allow optical expression detection, such as by way of fluorescent proteins. A multitude of suitable fluorescent proteins of different colors and other properties are known; examples include GFP, EGFP, CFP, YFP, DsRed, and HcRed, to name but a few. It is also preferred that the marker genes are controlled by suitable strong promoters, which have ideal characteristics to serve as transformation markers for a wide range of insect species (HORN (2002)). Examples include, but are not limited to the promoters PUb, 3×P3, actin5C and β2-tubulin.

In a further preferred embodiment, the transgenic insect according to the invention is homozygous for the first and the second gene expression cassette according to the invention, or is homozygous for the first or the second gene expression cassette.

In another preferred embodiment, the first and the second gene expression cassette and the first or the second gene expression cassette are further each comprised in a suitable vector construct. Generally, a suitable vector construct is any vehicle used to integrate foreign nucleic acid material into a genome, and typically contains elements that are capable of introducing, maintaining, and/or expressing nucleic acid sequences into a cell or, integrating nucleic acid sequences into the genome of a cell or of a host organism. Preferably, a suitable vector construct according to the invention further comprises an element selected from the group consisting of a transposon, a polytropic transposon, a retrovirus, a polytropic retrovirus, an element capable of homologous recombination, and an element capable of non-homologous recombination. Generally, a wide variety of suitable vectors are known in the art and available to the skilled person. Examples for suitable vectors comprising a transposon include, but are not limited to hobo, P, and Hermes. Examples for suitable vectors comprising a polytropic transposon include, but are not limited to piggyBac, Minos, and mariner. Examples for suitable vectors comprising a retrovirus are avian type C, BLV-HTLV, mammalian type B or C, and lentivirus retroviruses. Examples for suitable vectors comprising an element capable of homologous recombination with an insect's genome include, but are not limited to as described in RONG (2002). Examples for suitable vectors comprising an element capable of non-homologous recombination with the insect's genome include, but are not limited to Flp/FRT, Cre/lox, and phiC31/attP-attB containing vectors (WIMMER (2005)).

In a further preferred embodiment, the first gene expression cassette and the second gene expression cassette, or the first gene expression cassette or the second gene expression cassette, is/are located on chromosome 5 of Ceratitis capitata. Preferably, the first gene expression cassette and the second gene expression cassette are both located on chromosome 5 of Ceratitis capitata. The term “located on a chromosome” as used herein includes a stable integration of a nucleic acid element of a certain size into the nucleic acid sequence of a chromosome. The numbering of the chromosomes of C. capitata is effected according to ZACHAROPOULOU (1992). Methods for determining the location of inserted nucleic acid constructs on chromosomes of Ceratitis capitata are known to the skilled person and are described e.g. in ZACHAROPOULOU (1992).

In a further preferred embodiment, the first gene expression cassette or the second gene expression cassette according to the invention is located at a position selected from the group consisting of position 70B and position 63B of chromosome 5 of Ceratitis capitata. Preferably, the first gene expression cassette is at located position 70B, and the second gene expression cassette is located at position 63B of chromosome 5 of Ceratitis capitata or vice versa. In an even more preferred embodiment, the first gene expression cassette according to the invention is located at or near the nucleic acid sequence “ttaa” of chromosome 5 of Ceratitis capitata as identified by nucleotides no. 85-88 of SEQ ID NO: 13. In a further even more preferred embodiment, the second gene expression cassette is located at or near the nucleic acid sequence “ttaa” of chromosome 5 of Ceratitis capitata as identified by nucleotides no. 178-181 of SEQ ID NO: 12. Alternatively, either the first gene expression cassette or the second gene expression cassette is located at or near the respective positions above. By “located at” the sequence “ttaa” is meant that an insertion of a gene expression cassette of the invention occurs into the nucleic acid sequence “ttaa”. “Located near” the ttaa sequence of a position as used herein means that the insertion of a gene expression occurs near the respective positions above, but the gene expression cassette is still influenced by the genomic elements as if “located at”. A skilled person will be able to determine the site of insertion by standard techniques such as inverse PCR and DNA sequencing. Further, a skilled person will be able to effect the insertion of a given nucleic acid construct into certain sites of a chromosome of an insect and particularly into known sequence portions of chromosome 5 of Ceratitis capitata by using techniques for targeted modification of insect genomes such as homologous recombination, or by using a transposable element that integrates at “ttaa”-sites within an insect's genome. An example for a method of targeted introduction of DNA at specific sites within an insect's genome is described in RONG (2002), which reference is herewith incorporated in its entirety. An example for a transposable element that integrates at “ttaa”-sites within a genomic sequence is contained in the piggyBac vector as described in CARY (1989)

In another aspect, the invention provides a method of controlling reproduction in an insect population of interest, comprising the steps of (i) providing a plurality of insects according to the invention capable of interbreeding with the insects of the population of interest, (ii) optionally selecting suitable individual insects from the plurality, and (iii) allowing the insects of step (i) or (ii) to interbreed with insects of the population of interest. Preferably, the insects of step (i) or (ii) are released in an area where reproduction control of insects of the population of interest is desirable. The reproduction control is part and parcel of environmental-friendly area-wide insect pest management programs (AW-IPM). Examples for areas where such AW-IPM programs are applied include large farmland, huge plantations, or complete human residential areas

The term “controlling reproduction” of an insect population as used herein includes a directed influence on the number of offspring produced in any given insect population in a defined area. Preferably, reproduction control according to the methods of the invention results in a decrease of the number of offspring of an insect population of interest by infertile matings. Further preferred is that the reproduction control methods of the invention eventually result in the elimination, suppression, containment, or prevention of an insect population of interest or parts thereof in a defined area, and exclude a new introduction of such insects from other areas into the area of interest. For example, an eradication program has the ability to eliminate complete pest populations species-specifically and leads to a reduction in the use of insecticides, implying a long-term benefit for the environment. It can also be profitable to run a suppression program as an alternative to an eradication program in order to maintain the pest population below defined levels and ensure the economic health. Other examples are containment programs to protect neighboring pest free areas, which can be expanded gradually, or preventional programs avoiding the new establishment of invading exotic pests, or consolidating the progress made in an ongoing eradication program.

“An insect population of interest” as used herein means a number of insects of a particular species, typically living in a defined area such as contained in a laboratory or a rearing facility, or living in a given geographic area. Insect populations of interest are the targets of the developmental stage-specific lethality system of the invention. Of particular interest according to the invention are insect populations that act as pests in natural habitats, e.g. i) inflicting damage on crops, fruits, vegetables, animals, or humans, or ii) act as animal or human disease vectors.

“Providing a plurality of insects” as used herein means the provision of insects according to the invention in numbers and quality sufficient for the intended purpose of controlling reproduction in an insect population of interest. A method for producing transgenic insects comprising a developmental stage-specific lethality system according to the invention is set out below. Further methods of providing a plurality of insects by techniques such as breeding and rearing insects and evaluating their suitability for use in the methods of the invention are exemplified herein and known in the art.

In a preferred embodiment, insects or a plurality of transgenic insects of the invention are provided that further comprise a sexing system, preferably a genetic sexing system. In general, a sexing system allows the sex-specific elimination of individuals of an insect species, or disables individuals of an insect species in their reproductive capabilities in a sex-specific manner. In most cases, it is preferable that females are eliminated and male insects are selected from a plurality of insects before interbreeding with mates of a target insect population is allowed, which increases the efficiency of the method. Genetic sexing systems are known in the art, and include, e.g. transgenic sexing systems such as described in FU (2007), which is based on sex-specific splicing of a lethal effector, resulting in female-specific lethality. A further example of a genetic sexing system is the system based on the use of Y-linked transgenes described by CONDON (2007).

In a preferred embodiment, a genetic sexing system is used that is based on a temperature-sensitive lethal system in which individuals of a sex, preferably females, can be eliminated by exposure to elevated temperatures, as described in FRANZ (2005), allowing male insects to be selected from the plurality of insects according to the invention, e.g. for a subsequent release. In an advantageous embodiment, the genetic components making up genetic sexing systems are located on the same chromosome of the transgenic insect as the developmental stage-specific lethality system according to the invention. This would offer the advantage of facilitating the monitoring of the genetic status of insects used in methods of controlling reproduction before they are released into the environment. Particularly, it is desirable that all components of the genetic sexing system and the lethality system of the invention are located on chromosome 5 of Ceratitis capitata.

By “capable of interbreeding” with the insects of the population of interest is meant that the insects according to the invention that are used in a method of controlling reproduction according to the invention are capable of interbreeding, such as mating and producing fertilized eggs, with the insects of the population of interest that is to be controlled. Whether insects according to the invention are capable of interbreeding with insects of interest can be evaluated by the methods described herein, e.g. by the competition tests described in the Examples below. Such competition tests compare the reproductive success, i.e. the number of laid eggs versus the number of viable offspring, of insects according to the invention and wild type insects after competitive crossings. Typically, insects according to the invention are considered to be equally competitive with the insects of the population of interest if crossings using a ratio of 1 to 1 transgenic males to wildtype males lead to a measurable reduction in fertile eggs of about 50%, as e.g. described in the Examples below. Generally, transgenic insects according to the invention perform well in laboratory and field cage competition tests, which means that fewer individuals may have to be released in areas of population control in order to achieve the desired effect. From the laboratory competition tests, it is expected that when used in the context of a pest management program for population control, a ratio of released transgenic males to wildtype males ranging from 5:1 to 10:1 instead of the commonly applied 100:1 ratios in ongoing programs can be used.

Allowing the insects of the invention to interbreed with insects of the population of interest includes an interbreeding taking place e.g. under controlled conditions such as in a laboratory, or, preferably, by releasing the insects of the invention into a natural environment or an area where reproduction control of insects of the population of interest is desirable. Such a natural environment can be a geographical area of any size, e.g. large farmland, huge plantations, or complete human residential areas. The natural environment or area of interest may already be infested by the insect population that is to be controlled, or the area may be free of such insects but serve as a protective border to prevent the entry of a particular insect species from another inhabited area. Also, the area may be completely free of the insect pest but under constant threat of invasive species, an example of which would be the insect pest Ceratitis with regard to the Los Angeles Basin or Tampa, Fla.

In another aspect, the invention provides a method for producing transgenic insects comprising a developmental stage-specific lethality system comprising the steps of (i) providing a set of insects comprising a first gene expression cassette and/or a second gene expression cassette according to the invention, (ii) optionally subjecting the set of insects to one or more steps of interbreeding, (iii) evaluating the set of insects of step (i) or offspring obtained from the interbreeding steps of (ii) for functionality of the developmental stage-specific lethality system.

In a first preferred embodiment, the providing of a set of insects in step (i) can be achieved by providing a first set of insects comprising as first gene expression cassette according to the invention, then providing a second set of insects comprising a second gene expression cassette according to the invention, wherein insects from the second set are capable of interbreeding with insects of the first set. Insects comprising a first or second gene expression cassette according to the invention can be obtained by various transformation methods known in the art, e.g. by stable integration of DNA into the genome of the target species by way of electroporation, microinjection, biolistics, or lipofection using a suitable vector as described above carrying a gene expression cassette according to the invention. Typically, the gene expression cassettes integrate into the genome of the transformed insects e.g. by artificially induced transposition, homologous recombination or site-specific integration. Further methods for rearing and breeding insects obtained after transformation are known in the art and are e.g. described in the exemplifying section below.

In a second preferred embodiment, the first and the second gene expression cassette according to the invention are operably linked, e.g. linked in one contiguous DNA construct, and a set of insects comprising the operably linked construct is provided. Methods of obtaining transformed insects are known in the art and e.g. described above. In cases where the transactivating system comprised in the first and second gene expression cassette or the operably linked first and second gene expression cassette is already active when an insect is transformed with such a construct, it will be necessary to provide the insect with a suitable exogenous factor controlling the activity of the transactivating system before the insect is transformed with the construct. In case of the tTA system, this can be achieved e.g. by feeding the insect and/or its mother tetracycline or a derivative or an analogon thereof, before transformation is effected.

In a third preferred embodiment, it is contemplated that a first set of insects is provided comprising a first gene expression cassette according to the invention, and this first set of insects is then transformed with a second gene expression cassette according to the invention in a subsequent step. In an alternative embodiment, it is contemplated that the first or the second gene expression cassette is transformed into an insect, and the remaining (i.e. second or first respectively) gene expression cassette is subsequently integrated into the genome of the same insect in a directed fashion, for example by site-specific integration (e.g. using attP sites as described herein), or by homologous recombination, typically using regions homologous to corresponding portions of the genome that are suitable for a homologous recombination of a given construct with the genome. Furthermore, directed transposition events of one of the gene expression cassettes are contemplated, e.g. using transposable elements such as piggyBac or Minos.

Preferably, the transgenic insects used in the method for producing transgenic insects of the invention are as defined herein.

In a further aspect, the invention relates to the use of a transgenic insect according to the invention, or a transgenic insect obtainable by the methods according to the invention, for controlling reproduction in an insect population of interest, wherein the transgenic insect is capable of interbreeding with insects of the population of interest. Preferably, the transgenic insect is as defined herein. Further, the insect population of interest is as defined herein.

In another aspect, the invention provides a developmental stage-specific lethality system for use in a transgenic insect, comprising (i) a first gene expression cassette according to the invention, and (ii) a second gene expression cassette according to the invention, as defined herein. It is also preferred that the transgenic insect is as defined herein.

### DESCRIPTION OF THE FIGURES

FIG. 1. This figure shows examples of medfly genes expressed specifically during cellularization. Gene expression is shown by whole mount in-situ hybridization (WMISH) with gene-specific RNA probes for different stages during embryogenesis: early blastoderm (×1), cellularization (×2), germ band elongation (×3) and germ band retraction (×4). The genes C.c.-slam (Ay), C.c.-sub2_{—}99 (By), C.c.-CG2186 (Cy), C.c.-sry α (Dy), C.c.-sub2_{—}63 (Ey), and C.c.-sub2_{—}65 (Fy) are strongly expressed during cellularization (×2). C.c.-sub2_{—}63 showed also expression during germ band elongation (E3).

FIG. 2. This figure shows the tTA and hid^{Ala5} expression under control of different promoter/enhancers (P/Es). Expression of tTA and hid^{Ala5} is shown by WMISH performed on embryos from medfly lines carrying both driver and effector constructs in homozygous condition. The embryogenesis is pictured by early blastoderm (×1 and ×4), cellularization (×2 and ×5), and germ band elongation/retraction (×3 and ×6). The lines carry driver constructs with different P/E (P) driving the tTA. The depicted lines are representative for independent lines (three for sl1, two for sl2, three for 99, and one for CG2186) carrying the respective driver construct. All presented lines derive from the effector line TREhs43-hid^{Ala5}_F1 m2 and were reared on Tc-free adult food. 100% lethality in lab tests is indicated with +, and the stage of complete lethality is indicated in brackets.

FIG. 3. This figure shows tTA and hid^{Ala5} expression at different integration sites. The expression of tTA and hid^{Ala5} is shown by WMISH performed on embryos from medfly lines carrying both driver (D) sryα2-tTA with the sry α P/E element driving the tTA and effector (E) TREhs43-hid^{Ala5} in heterozygous conditions. Independent integrations of driver and/or effector construct are indicated in brackets. Lines were reared on Tc-free adult food for this experiment. 100% lethality in lab tests is indicated with +, and the stage of complete lethality is indicated in brackets.

FIG. 4. This figure shows Southern hybridizations of BamHI-digested genomic DNAs (A and B) isolated from indicated medfly lines, hybridized with DsRed (A) or EGFP (B) probes, respectively. WT genomic DNA was used as a control for both. A single band in each lane indicates single integrations of the transgenes.

FIG. 5. This figure depicts efficiency, competition and reversibility tests with strains carrying the controllable lethality system according to the invention. (A) Efficiency test: The adult progeny of virgin WT females crossed to males from lines #29, #72, #66, #67, #68, or WT are displayed, respectively. For each line, four independent repetitions of 24 h egg collections were taken five days after crossing. Tc-free adult and larval food was used. Hatched L1 larvae 48 h after egg collection (black bars), total pupae (white bars), and total adults (grey bars) were counted, and are shown in relation to the total number of eggs from four independent egg collections (total egg number: n (#29)=1481; n (#72)=4330; n (#66)=2278; n (#67)=2058; n (#68)=1914; n (WT)=1712). Due to difficulties in the larval count, the number of surviving larvae might be an under-representation. The SD of two repetitions is indicated. Repetitions are non-significantly different (ns), shown by t-tests (Table 1).

(B) Competition for virgin WT females: 15 WT females and 15 WT males were placed together with different numbers of #66 or #67 males (15 (1:1:1)-135 (1:1:9)). For control matings, 15 virgin WT females were crossed with either 15 WT males (+) or 150 WT males (++). Six 24 h egg collections were performed from two repetitions for each independent crossing and the number of adult progeny was recorded. Numbers are normalized to positive control (+). The SD of two repetitions is indicated. Repetitions are ns, shown by t-tests (Table 1).

(C) Reversible lethality: Three day old flies from #66 (grey bars) and #67 (black bars) were reared on Tc-containing food (+Tc; 10 μg/ml) for two days, transferred to Tc-free medium (−Tc) for five days and transferred back to Tc-containing food for three days. Progeny of 24 h egg lay intervals were monitored (embryos collected and emerging adults scored). The ratio of adults to laid eggs is shown. The SD of two repetitions is indicated. Repetitions are ns, shown by chitest (Table 1).

FIG. 6. Chromosome in-situ hybridization on polytene chromosome spreads of embryonic lethality line (LL) #67. A double in-situ hybridization on spread chromosomes from LL #67 is shown. The two integration sites of driver construct sryα2-tTA_PUbDsRed and effector construct TREhs43-hid^{Ala5} PUbEGFP were recognized at positions 5L_{—}63B and 5L_{—}70B. This type of detection does not allow us to decide which construct is at which integration site.

FIG. 7. Schematic representation of chromosome 5 from the embryonic LL #67. The two arrows show the integration sites of the effector construct TREhs43-hidAla5_PUbEGFP and the driver construct sryα2-tTA_PUbDsRed in respect to other genetic markers on the fifth chromosome. The centromere is indicated as C.

FIG. 8. Mating competitiveness of line #67 in field cage tests. To test the competitiveness of the embryonic lethal line #67, 20 non-irradiated and 20 irradiated males from line #67 competed with 20 non-irradiated wild type Argentinean (Arg) males for mating with 20 wild-type Argentinean females in a field cage. The males were marked with different colored water-based paints. Mating couples were taken out of the cage and the type of mating couple was recorded. Twelve replications were carried out. (A) The proportion of matings (PM) of each mating type was calculated by dividing the number of the occurred matings by the number of total possible matings (limited by the number of Argentinean females, n=20). The proportion of matings was 18±11% for non-irradiated #67 males, 13±9% for irradiated #67 males and 12±12% for non-irradiated Argentinean males. The proportion of total matings over all twelve replications was 43±5% indicating an acceptable degree of sexual activity during the test period. The tests showed that non-irradiated and irradiated #67 males were at least as, if not more competitive than wild type non-irradiated Argentinean males. (B) Eggs and hatched larvae from each mating type were recorded and the egg hatch is shown. All matings of #67 males (regardless whether non-irradiated or irradiated) to wild type Argentinean females led to complete embryonic lethality.

In comparison to the complete lethality of strain #67 (descending from EgII) with or without irradiation, previous sterility tests with irradiated wild type EgII males (100 Gy) showed an egg hatch of 1.2% FRANZ (2000). In addition, radiation induced sterility has been shown to be indirectly correlated to the competitiveness of the flies PARKER and MEHTA (2007).

### EXAMPLES

The following examples are meant to further illustrate, but not limit, the invention. The examples comprise technical features, and it will be appreciated that the invention relates also to combinations of the technical features presented in this exemplifying section.

### Example 1

### Isolation of Cellularization-Specifically Expressed Genes and their P/Es from Medfly (C. capitata)

The Clontech PCR-Select cDNA Subtraction Kit (BD Biosciences, Heidelberg) was used to isolate fragments of the following genes expressed specifically during cellularization according to the techniques described in SCHETELIG (2007), which reference is herewith incorporated in its entirety: C.c.-slam, C.c.-sub2_{—}99, C.c.-CG2186, C.c.-sub2_{—}63, and C.c.-sub2_{—}65. An EST fragment of the medfly cellularization gene serendipity α (C.c.-sry α) was received from Dr. Ludvik Gomulski, Pavia. By RACE, 5′ and 3′ ends of cellularization specific genes were isolated using the BD SMART RACE cDNA Amplification Kit (BD Biosciences, Heidelberg) and gene specific primers. Complete cDNA sequences are shown in SEQ ID NO. 6-11.

Inverse PCR was performed to obtain the 5′ regions of genes specifically expressed during cellularization: 1.5 μg of medfly WT genomic DNA was digested for 24 h; restriction fragments were precipitated and self-ligated in a volume of 500 μl at 16° C. for 24 h; PCR was performed on circularized fragments by using primer sequences in opposite orientation within the 5′UTR or ORF of the genes. First PCRs (1 min at 95° C.; 6 cycles of 30 sec at 94° C., 45 sec at 66° C. (−2° C. each cycle), 6 min at 68° C.; 25 cycles of 30 sec at 94° C., 45 sec at 54° C., 6 min at 68° C.; and 6 min at 68° C.) for C.c.-slam, C.c.-sub2_{—}99, C.c.-CG2186, C.c.-sry α or C.c.-sub2_{—}63 were performed on FspBI, NdeI, CviAII, PvuI or AcII cut genomic DNA with the primer pairs mfs-77/-79 (SEQ ID NO. 18 and 20), mfs-85/-108 (SEQ ID NO. 23 and 25), mfs-170/-172 (SEQ ID NO. 41 and 43), mfs-159/-161 (SEQ ID NO. 37 and 39) or mfs-83/-104 (SEQ ID NO. 22 and 24), respectively, using BD Advantage 2 PCR (BD Biosciences, Heidelberg). Second, the obtained PCR products were diluted 1:50 with ddH₂0 and nested PCRs with primer pairs mfs-78/-80 (C.c.-slam, SEQ ID NO. 19 and 21), mfs-160/-162 (C.c.-sry α, SEQ ID NO. 38 and 40), or mfs-171/-173 (C.c.-CG2186, SEQ ID NO. 42 and 44) were performed (1 min at 95° C.; 22 cycles of 30 sec at 94° C., 45 sec at 54° C., 6 min at 68° C.; and 6 min at 68° C.) using 5 μl of the dilution and the BD Advantage 2 PCR Kit (BD Biosciences, Heidelberg). PCR products from first (C.c.-sub2_{—}99 and C.c.-sub2_{—}63) and nested PCRs (C.c.-slam, C.c.-sry α and C.c.-CG2186) were cloned into pCRII vectors (Invitrogen, Karlsruhe) and sequenced.

### Example 2

### Construction of the Driver Constructs

Generally, constructs were prepared in the cloning shuttle vector pSLfa1180fa. From the shuttle vectors, the constructs can be easily placed in transformation vectors, which carry FseI and AscI sites (fa-sites; HORN AND WIMMER (2000)).

The pSLaf_attP-sl2-tTA_af (#1231), pSLaf_attP-63-tTA_af (#1232), pSLaf_attP-99-tTA_af (#1234), pSLaf_attP-sryα2-tTA_af (#1236) and pSLaf_attP-ccCG2186-tTA_af (#1237) carry a 52 bp attP site (THORPE (2000)). #1231, #1232, or #1234 was created by ligating annealed attP primers (mfs-201/-202, SEQ ID NO. 49 and 50) in the EcoRI cut pSLaf_sl2-tTA_af (#1210), pSLaf_{—}63-tTA_af (#1211) or pSLaf_{—}99-tTA_af (#1212), respectively. #1236 or #1237 was created by ligating annealed attP primers (mfs-203/-204, SEQ ID NO. 51 and 52) in the NcoI cut pSLaf_sryα2-tTA_af (#1225) or pSLaf_CG2186-tTA_af (#1226), respectively.

#1210, #1211, or #1212 was created by ligating the EcoRI-XbaI cut sl2 fragment (a 1.9 kb 5′-region of the gene C.c.-slam), the EcoRI-Eco31I cut 63 fragment (a 1.2 kb 5′-region of the gene C.c.-sub2_{—}63) or the EcoRI-XbaI cut 99 fragment (a 0.7 kb 5′-region of the gene C.c.-sub2_{—}99), amplified by PCR on genomic DNA with primer pairs mfs-141/-113 (SEQ ID NO. 34 and 29), mfs-142/-143 (SEQ ID NO. 35 and 36), or mfs-131/-133 (SEQ ID NO. 32 and 33), in the EcoRI-XbaI cut pSLaf_tTA_af (#1215), respectively. #1225 or #1226 was created by cloning the NcoI-XbaI cut sryα2 fragment (a 1.6 kb 5′-region of the gene C.c.-sryα) or the NcoI-Eco31I cut CG2186 fragment (a 1.2 kb 5′-region of the gene C.c.-CG2186), amplified with primer pairs mfs-189/-188 (SEQ ID NO. 45 and 46), or mfs-190/-191 (SEQ ID NO. 47 and 48), in the NcoI-XbaI cut #1215, respectively. #1215 was generated by cloning a 1.5 kb XbaI-HindIII cut tTA-SV40 fragment from pTetOff (Clontech, CA) in the XbaI-HindIII cut pSLfa1180fa (HORN AND WIMMER, 2000).

The driver construct pBac{sl1-tTA_PUb-DsRed} (sl1-tTA) was generated by ligating the BglII/XbaI cut sl1 (a 0.4 kb 5′-region of the gene C.c.-slam amplified with primer pair mfs-112/-113 (SEQ ID NO. 28 and 29) from genomic DNA) and the XbaI/BglII cut tTA-SV40 (a 1.5 kb region amplified with primer pair mfs-110/-111 (SEQ ID NO. 26 and 27) from pTetOff) in the BglII site of pB[PUbDsRed1] (HANDLER AND HARRELL (2001)).

The driver constructs pBac{f_attP-sl2-tTA_a_PUb-DsRed} (sl2-tTA), pBac{f_attP-63-tTA_a_PUb-DsRed} (63-tTA), pBac{f_attP-99-tTA_a_PUb-DsRed} (99-tTA), pBac{f_attP-sryα2-tTA_a_PUb-DsRed} (sryα2-tTA) or pBac{f_attP-CG2186-tTA_a_PUb-DsRed} (CG2186-tTA) were generated by ligating the FseI-AscI fragment attP-sl2-tTA, attP-63-tTA, attP-99-tTA, attP-sryα2-tTA or attP-CG2186-tTA from #1231, #1232, #1234, #1236 or #1237 in the FseI-AscI cut pBac{fa_PUb-DsRed} (#1200), respectively.

#1200 or pBac{fa_PUb-EGFP} (#1201) were created by cloning hybridized primers mfs-117/-118 (SEQ ID NO. 30 and 31) in the BglII site of pB[PUbDsRed1] or pB[PUbnlsEGFP] (HANDLER AND HARRELL (1999)), respectively.

By inverse PCR, the P/Es from C.c.-slam, C.c.-sub2_{—}99, C.c.-CG2186, C.c.-sry α and C.c.-sub2_{—}63 containing about 0.4 to 1.9 kb of 5′UTR and upstream sequences were isolated. The isolated P/Es were fused to the tetracycline-controlled transactivator gene tTA and used to engineer different driver constructs (sl1-tTA, sl2-tTA, 99-tTA, CG2186-tTA, sryα2-tTA and 63-tTA) embedded into piggyBac vectors carrying polyubiquitin (PUb) driven DsRed as germline transformation marker (HANDLER AND HARRELL (2001).

### Example 3

### Construction of the Effector Constructs

The effector constructs pBac{fa_attP_f_TREp-hid^{Ala5}_a_PUb-EGFP} (TREp-hid^{Ala5}) or pBac{fa_attP_f_TREhs43-hid^{Ala5}_a_PUb-EGFP} (TREhs43-hid^{Ala5}) were generated by cloning the hybridized primers mfs-211/-212 (SEQ ID NO. 53 and 54) in the XmaJI site of pBac{faf_TREp-hid^{Ala5}_a_PUb-EGFP} (#1207) or pBac{faf_TREhs43-hid^{Ala5}_a_PUb-EGFP} (#1208), respectively. #1207 or #1208 were created by ligating the AscI fragments TREp-hid^{Ala5} (5.0 kb) or TREhs43-hid^{Ala5} (4.9 kb) from pSLfa_TREp-hid^{Ala5}_fa or pSLfa_TREhs43-hid^{Ala5}_fa (HORN AND WIMMER (2003)) in the AscI site of pBac{fa_PUb-EGFP} #1201 (SCOLARI (2008)), respectively. The effector construct pBac{>fa_attP_f_TREp-hid^{Ala5}_a>_PUb-EGFP} (>TREp-hid^{Ala5}>) was generated by ligating the AscI-fragment attP_f_TREp-hid^{Ala5} from TREp-hid^{Ala5} in the AscI-site of pBac{>fa>_PUb-EGFP} (SCOLARI (2008)).

Three effector constructs were generated (TREp-hid^{Ala5}, TREhs43-hid^{Ala5}, and >TREp-hid^{Ala5}>) carrying the lethal factor hid^{Ala5} under control of either p or hsp70 basal promoters from D.m. In the >TREp-hid^{Ala5}> construct the lethality inducing transgene is flanked by gypsy insulator elements (>=gypsy element in 5′-3′ orientation), which should reduce the variable expression strength due to position effects (SARKAR (2006)). Except for sl1-tTA, all constructs carry a minimal attachment P (attP) site, which will potentially enable site-specific integration to modify the transgenic situation.

### Example 4

### Germline Transformation with Driver and Effector Constructs

WT and transgenic medfly lines were maintained under standard rearing conditions (SAUL (1982)). The WT strain Egypt-II was obtained from the FAO/IAEA Agriculture and Biotechnology Laboratory (Seibersdorf, Austria).

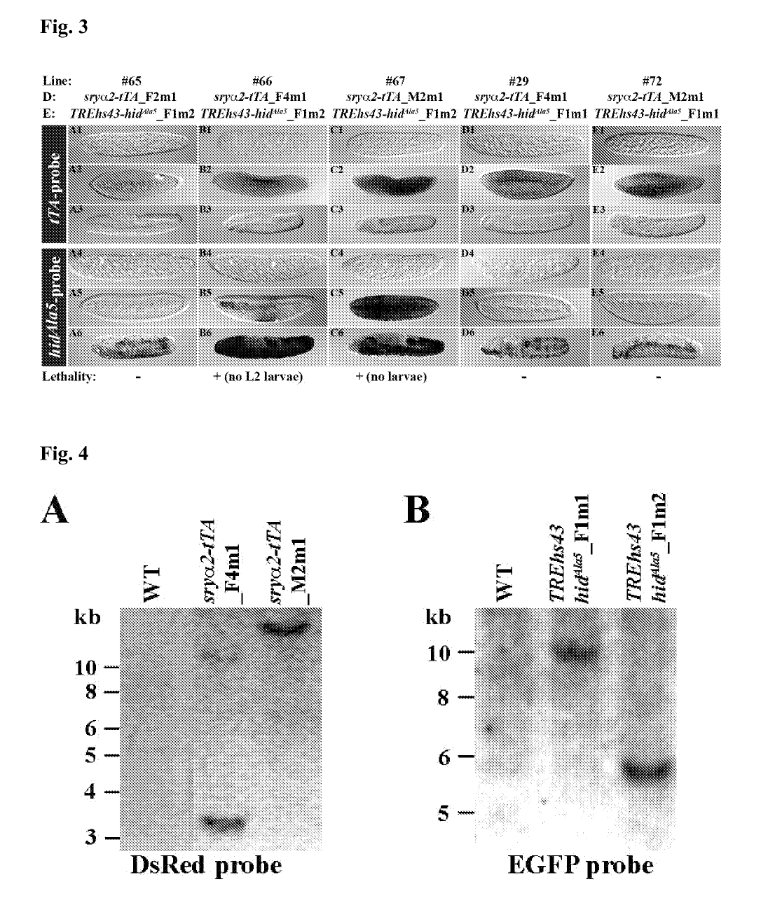

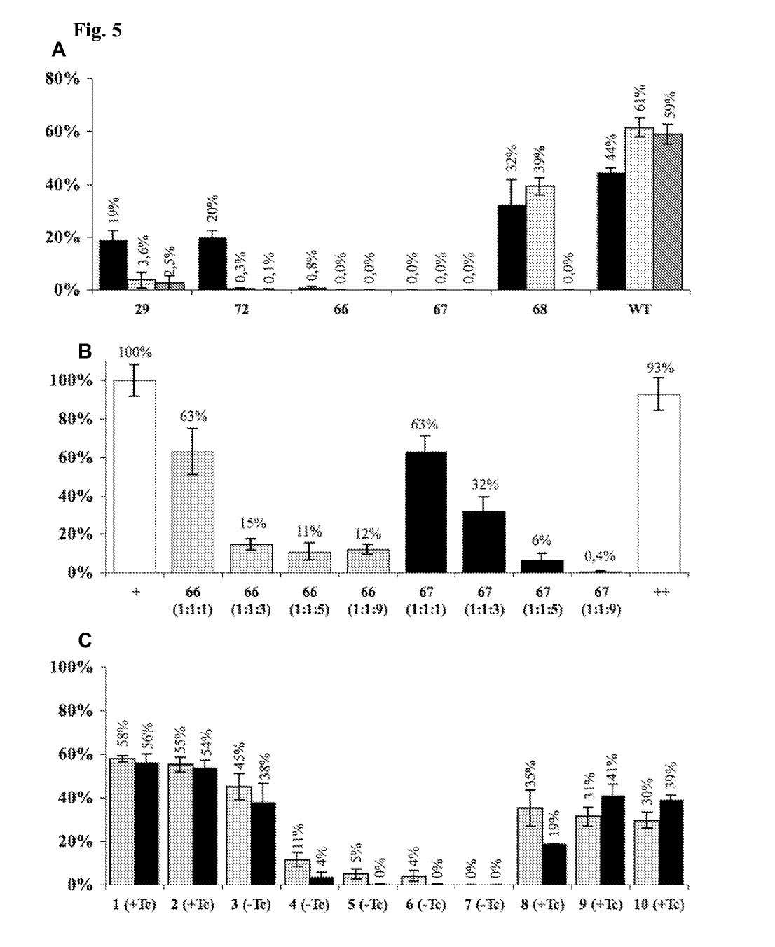

Five driver constructs (sl1-tTA, sl2-tTA, 99-tTA, CG2186-tTA and sryα2-tTA) and all three effector constructs were used for germline transformation of medfly. The vectors sl1-tTA, sl2-tTA, 99-tTA, sryα2-tTA, CG2186-tTA, TREp-hid^{Ala5}, TREhs43-hid^{Ala5}, or >TREp-hid^{Ala5}> were injected into 600 embryos of which 260, 140, 160, 54, 83, 28, 63, or 52 survived to adulthood, respectively. Four female crossings (two to 25 G₀ females crossed to 15 WT males; F1-F4) and four male crossings (two to 25 G₀ males crossed to 15 WT females; M1-M4) were set up for each construct. G1 progeny were screened by epifluorescence for the expression of the PUb-DsRed or PUb-EGFP. Fluorescent progeny with different red or green patterns were backcrossed twice to WT to recognize possible multi-insertions and brought to homozygous conditions by inbreeding and checking fluorescence intensity. For each construct we obtained transgenes of which we further analyzed a maximum of three independent lines (FIG. 2 and FIG. 3).

Germline transformation experiments were performed by microinjection of piggyBac constructs (500 ng/μl) together with the phspBac transposase helper plasmid (200 ng/μl) (HANDLER AND HARRELL (1999)) into WT embryos as described by HANDLER AND JAMES (2000) with the following exceptions: injected eggs were covered with Voltalef 10S oil (Lehmann & Voss, Hamburg, Germany), placed at 28° C. in parafilm closed Petri dishes with watered Whatman paper in the lid; neither eggs, larvae or pupae were heat shocked; enclosed G0 males and virgin females were backcrossed in groups of 1-3 individuals to 5-15 virgin WT females or five WT males, respectively. G1 progeny were screened by epifluorescence for the expression of the PUb-DsRed or PUb-EGFP. For screening and images of flies the fluorescence stereomicroscope Leica MZ16 FA with the filters DsRedwide (Ext. 546/12; Emm. 605/75) and EYFP (Ext. 500/20; Emm. 535/30) was used. Images were taken with an Intas MP Focus 5000 digital camera.

To generate lethality lines, twelve homozygous driver lines and five homozygous effector lines were crossed to generate 60 different combinations. From each combination, eggs were collected to visualize the early expressed tTA and the proapoptotic gene hid^{Ala5} by in-situ hybridizations. The lethal activity of each combination was checked by a second egg collection, which was counted for eggs and progeny. To describe the dimension of lethality, the term “complete lethality” is henceforth used for 100% lethality in laboratory experiments. Combinations that showed detectably lower or no progeny were inbred to generate homozygous (for both driver and effector construct) lethality lines (LLs).

All LLs expressed tTA specifically during cellularization. However, due to the different P/Es the tTA expression strength varied and resulted in different expression strengths of hid^{Ala5} (FIG. 2 and FIG. 3). This resulted in variable efficiencies of the lethality system. LLs deriving from the same driver line showed always similar expression levels of tTA. The P/Es sl1 (FIG. 2A1-3) and 99 (FIG. 2C1-3) mediated only very weak expression of tTA, which subsequently could not induce detectable levels of hid^{Ala5} expression. The longer P/E region of sl2 (1.9 kb) was able to drive tTA and indirectly hid^{Ala5} (FIG. 2B1-6), but the level of hid^{Ala5} expression was not enough to drive complete lethality (FIG. 2). With the P/E CG2186 we obtained a very strong level of tTA expression during cellularization (FIG. 2D2), which started the expression of hid^{Ala5} during the cellularization stage (FIG. 2D5) and led to complete pupal lethality of LL #68 (FIG. 2).

Besides the finding that different P/Es or P/E regions act differently on tTA and the dependent hid^{Ala5} expression, also the integration site of the driver construct could influence the tTA expression (FIG. 3). Three independent lines, carrying the driver construct with the sry α P/E at different integration sites, expressed the tTA specifically but with different strength during cellularization (FIG. 3A2-C2). In line #65, a weak expression of tTA led to a late expression of hid^{Ala5} during germ band retraction, which was not able to drive complete lethality (FIG. 3). In contrast, the lines #66 and #67 express tTA strongly during cellularization (FIG. 3B2, C2), which activates hid^{Ala5} expression in the cellularization stage (FIG. 3B5, C5) and lead to complete L1 larval lethality for line #66 and complete embryonic lethality for line #67 (FIG. 3).

In addition, also the effector constructs with different basal D.m. promoters or different integrations of the same effector construct might influence the levels of hid^{Ala5} expression and lethality. The effector constructs TREp-hid^{Ala5} and >TREp-hid^{Ala5}>, carrying the p-basal promoter, were able to express hid^{Ala5} in medfly after activation through the twelve independent driver lines, but did not cause complete lethality in 36 different LLs (data not shown). Interestingly, the effector construct TREhs43-hid^{Ala5}, which carries the basal promoter (43 bp) of hsp70, showed differences in the expression strength of hid^{Ala5} depending on the integration site of the construct. In comparison to the larval or embryonic lethal lines #66 or #67, which are derived from the effector line TREhs43-hid^{Ala5} F1m2, the hid^{Ala5} expression in #29 and #72 deriving from TREhs43-hid^{Ala5}_F1m1 started during germ band elongation/retraction (FIG. 3D6, E6) and was not sufficient to drive complete lethality at the larval, pupal or adult stage.

### Example 5

### Southern Hybridization

Genomic DNA (˜3-10 μg) from adult flies of different transgenic lines and the WT strain were digested with BamHI (Roche, Mannheim, Germany) and separated on 1% agarose gels. DNA was transferred to nylon membranes (Hybond-N+; Amersham Biosciences) and immobilized by UV irradiation. Probe labeling and membrane hybridizations were performed according to the AlkPhos Direct kit (GE Healthcare, Little Chalfont, UK). Signal detection was performed using CDP-star (GE Healthcare, Little Chalfont, UK) followed by exposure for approximately 30 min on Kodak Biomax ML film.

The two probes for detecting DsRed or EGFP were amplified by PCR (2 min at 94° C.; 30 cycles of 30 sec at 94° C., 30 sec at 53° C., 1 min at 72° C.; 5 min at 72° C.) from the constructs #1200 or #1201 with the primers mfs-333 (SEQ ID NO. 57) and mfs-334 (SEQ ID NO. 58) or mfs-335 (SEQ ID NO. 59) and mfs-336 (SEQ ID NO. 60), respectively.

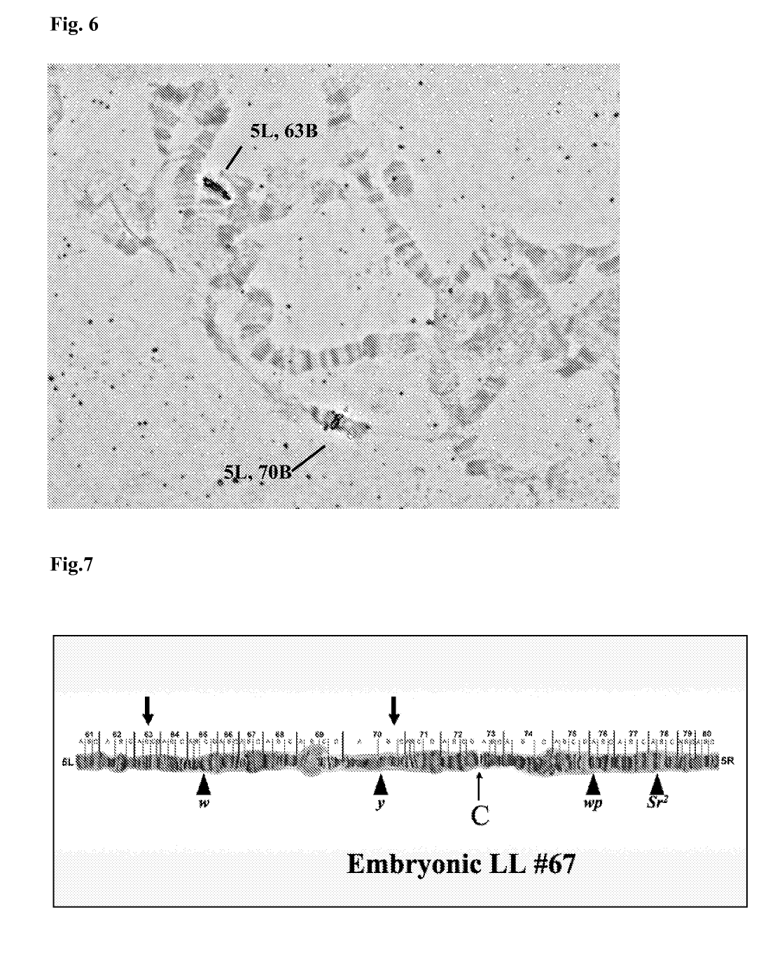

Southern blots to BamHI-digested genomic DNA of the driver lines sryα2-tTA_F4m1 and sryα2-tTA_M2m1 using a DsRed-specific probe (FIG. 4A) and of the effector lines TREhs43-hid^{Ala5}_F1m1 and TREhs43-hid^{Ala5}_F1m2 using an EGFP-specific probe (FIG. 4B) indicated single copy integration of the respective constructs. Moreover, the correct piggyBac-mediated integrations at canonical TTAA target sites were verified by isolation of 5′ and 3′ insertion site sequences by inverse PCR. Therefore we know, that differences in expression strength and functionality of the lethality system in different LLs are not a result of multiple insertions of the driver or effector constructs, but must be due to position effects.

### Example 6

### Genomic Localization of Integrations

Furthermore, the integration sites of the driver and effector construct for LL #67 were mapped by chromosome spreads. The driver and the effector were located on chromosome 5 at the positions 63B and 70B (FIGS. 6 and 7). The detection method described in the next paragraph does not allow us at the moment to decide which construct is at which integration site.

Chromosome in-situ hybridizations and detection of labelled DNA were performed with slight modifications as described (Zacharopoulou et al, 1992). Instead of horseradish peroxidase, the Biotin/Avidin system VECTASTAIN Elite ABC was used (Vector laboratories, Peterborough). Hybridization sites were identified and photographed using 60× oil objectives (Olympus phase contrast microscope) with reference to medfly salivary gland chromosome maps (Gariou-Papalexiou, 2002). Squash preparations of salivary gland polytene chromosomes were made as described (Zacharopoulou et al, 1992). A DNA-probe, recognizing as well DsRed as EGFP constructs, was prepared by PCR on genomic DNA from flies carrying a DsRed construct (Handler and Harrell, 2001) with the primers DsRed_F (SEQ ID NO. 55) and DsRed_R (SEQ ID NO. 56) (1813 bp) using the Biotin High-Prime kit (Roche Diagnostics, Mannheim).

The additional finding that both constructs of the embryonic lethal and competitive line #67 are located on chromosome 5 has several advantages. First, this line can be combined with different well established systems carrying their effectors also on chromosome 5: e.g. the phenotypic marker system sr² (NIYAZI (2005)) or genetic sexing strains (GSSs) like Vienna-8 (FRANZ (2005)) with the wp marker and the tsl-mutation both on chromosome 5. The advantage of having different systems on chromosome 5 is a simplified quality control during rearing procedures. Second, the embryonic lethality line provides two fluorescent markers (DsRed and EGFP), which are not only helpful during quality control but could also help during monitoring processes. Third, the constructs introduced attP sequences, which will allow site-specific modification of this competitive embryonic LL by using the integrase system from phage phiC31, (GROTH (2004)). Possible applications will be the deletion of piggyBac ends to further increase the safety of transgenes or insertion of recently developed sperm markers for improved monitoring (SCOLARI (2008)).

To localize the integration sites of piggyBac vectors, inverse PCR was performed with primers and protocols as described in HORN 2003. Sequences flanking piggyBac insertions are shown e.g. in SEQ ID NO. 12 and in SEQ ID NO. 13, which are located at positions 5L-70B or 5L-63B of chromosome 5 of C. capitata.

### Example 7

### In-Situ Hybridization

WMISH with RNA probes to embryos were performed as described (Davis et al., 2001). RNA antisense probes were prepared by in-vitro transcription with the DIG-RNA-Labeling Kit (Roche, Mannheim) from pCRII vectors (Invitrogen, Karlsruhe) containing subtraction cDNA fragments (p_slam, p_{—}99, p_CG2186, p_{—}63, p_{—}65), an EST fragment (p_sryα) and the plasmids pBSK-hid^{Ala5} or pBSK-tTA (HORN AND WIMMER (2003)). By PCR using the primer pair mfs-41/-42 (SEQ ID NO. 61 and SEQ ID No. 62), cDNA fragments were amplified and transcribed with Sp6 polymerase. The plasmids pBSK-hid^{Ala5} or pBSK-tTA were linearized with ClaI or EcoRI and transcribed with T3 or T7 RNA polymerase, respectively.

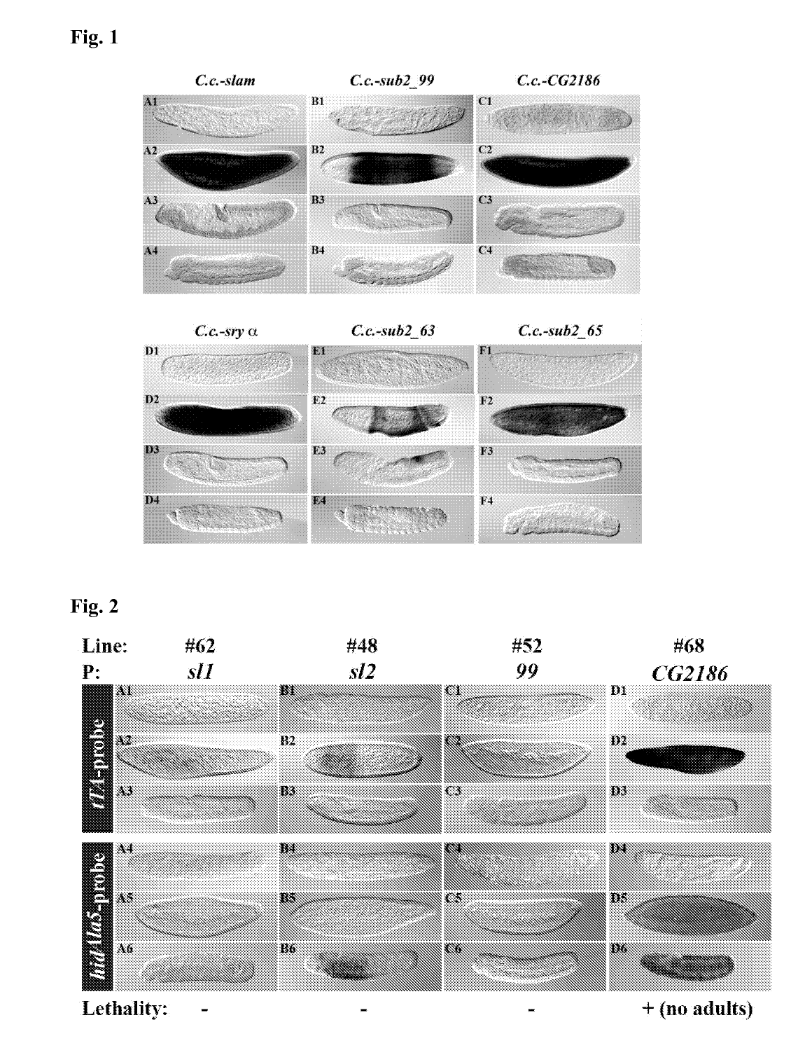

PCR-based cDNA subtractions of different embryonic stages identified several cellularization-specific genes (FIG. 1). The genes C.c.-slow as molasses (C.c.-slam; FIG. 1A), C.c.-sub2_{—}99 (FIG. 1B), C.c.-CG2186 (FIG. 1C), C.c.-serendipity α (C.c.-sty α; FIG. 1D), C.c.-sub2_{—}63 (FIG. 1E), and C.c.-sub2_{—}65 (FIG. 1F) are expressed specifically during medfly blastoderm cellularization (FIG. 1x2). C.c.-sub2_{—}63 is additionally expressed during germ band elongation (FIG. 1E3). None of the genes show maternal expression or expression at the gastrulation stage. Thus, the P/Es of these genes can be used for driving a lethality system without interfering with the adult phase of the medfly life cycle or with gametogenesis.

### Example 8

### Optimization of Tc-Concentrations for Rearing

Starting from larval and adult media Tc-concentrations of 100 μg/ml, the minimal Tc concentrations for the lines #29, #66, #72, #67, and #68 were tested with WT as a control. Firstly, flies were reared on adult medium containing 100 μg/ml Tc and eggs were collected on larval medium containing 0, 1, 3, 10, 30, 100, or 300 μg/ml Tc. Hatching, pupation and eclosion rates were recorded. Second, the adult medium Tc concentrations (1, 3, 10, 30, 100, or 300 μg/ml) were tested over three generations by using the optimized larval media concentrations (1 μg/ml for #29, #66, #72, and #67; 10 μg/ml for #68) in between the adult stages.

To identify the minimal concentrations of Tc needed to rear the LLs #29, #72, #66, #67, and #68, flies were bred on larval and adult media containing different concentrations of Tc. The optimal Tc concentration in adult and larval medium for rearing as the lowest possible amount of Tc combined with the highest possible number of descendants were defined. The LLs #72, #66, or #67 could be reared efficiently on adult medium containing 10 μg/ml Tc and line #29 even on 1 μg/ml Tc. All LLs could be reared on larval medium containing 1 μg/ml Tc, except for #68 (10 μg/ml Tc). Using larval medium lacking Tc or Doxycycline lines #66 and #67 showed maternal suppressibility. When reared on larval medium containing 300 μg/ml Tc, all lines and WT showed slowed down ovary development and a 5-7 days postponed egg laying. This indicates the importance of reducing the Tc concentrations to a minimum for the efficient rearing of medfly lines.

Today, mass rearing facilities like El Pino in Guatemala need tons of larval food daily to rear the larvae. For suppression of lethality during the mass rearing, Tc or Doxycycline would be a supplement in the adult and/or larval food. By using the optimal Tc concentrations for rearing, the complete lethality system is switched off and in case that only adult food contains the supplement, tons of larval food can be reused for fish farming or cattle breeding after the mass-rearing process.

### Example 9

### Efficiency of the Lethality System

Twelve independent medfly driver lines were crossed to five independent effector lines. All lines, which produced detectably lower or no progeny (#29, #72, #66, #67, and #68) on Tc-free food, were further characterized. In four independent repetitions, homozygous males from #29, #72, #66, #67, or #68 were crossed to virgin WT females on Tc-free food directly after eclosion. Four days later a 24 h egg collection was taken. Eggs, L1 larvae (48 h after egg collection), pupae, and adults were scored.

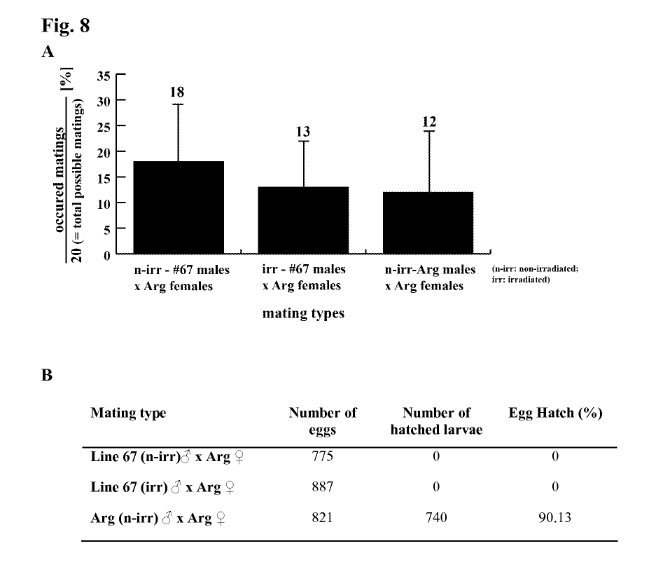

During medfly SIT programs, irradiation-sterilized males are released into affected areas and mate to WT females, which leads to infertile matings. Ideally all progeny die as embryos to exclude damage to fruits from larval feeding. To show the efficacy and time point of lethality for the newly generated LLs, transgenic males (homozygous for driver and effector) from lines #29, #72, #66, #67, #68, or WT were crossed to WT females, respectively (FIG. 5A). For the LLs #29 and #72, about 20% of the eggs survived to become L1 larvae, whereas pupae and adult progeny are highly reduced. Crossings with males from #66, #67, and #68 showed complete pupal lethality besides varying larval and embryonic lethality. Only 0.8% of the laid eggs from the #66-crossing hatched and all of those died during L1 larval stage. Line #67 showed the desired complete embryonic lethality.

### Example 10

### Laboratory Competition Test

Freshly enclosed 15 WT females and 15 WT males were crossed together with different numbers of males from lines #66 or #67 (tested ratios: 1:1:1, 1:1:3, 1:1:5, 1:1:9). For control matings, 15 WT females were crossed to 15 WT males (+) or 150 WT males (++). Eggs were collected for one week every 24 h. Adult progeny were counted and verified by fluorescence light microscopy as WT or transgenic offspring. Two independent crossings were performed for each ratio of both transgenic lines.

An ideal line for releasing purposes should be embryonic lethal, but should also be competitive. Therefore, competition tests were done with lines #66 and #67 (FIG. 5B). WT females were crossed to WT males and transgenic males in different ratios (1:1:1, 1:1:3, 1:1:5, 1:1:9). The overall progeny compared to the laid eggs showed that both strains were highly competitive to WT. Remarkable was the higher fertilization success of #67 males compared to WT males starting from ratio 1:1:5. For the ratio 1:1:9 an overall progeny rate of only 0.4% was measured (10% are expected, for equal competitiveness). At the same time, a WT control at ratio 1:10:0 gave only little reduction of overall progeny (FIG. 5B, ++). Transgenic males from line #66 and #67 performed in laboratory competition tests comparable or even better than WT males. Progeny from all competition tests were identified as non-transgenic individuals by fluorescent microscopy, which additionally indicated the complete lethality of line #66 and #67. Interestingly, all lines deriving from the effector line TREhs43-hid^{Ala5}_F1m2 (#66, #67, and #68) partially lack anterior orbital bristles, which does obviously not interfere with the mating success of these transgenic males. In addition to laboratory tests, field cage tests with #67 males showed a comparable or even better competitiveness than wild type Argentinean males (see FIG. 8 and Example 11). Thus, the lines showing complete embryonic lethality are also highly competitive to wildtype medfly in laboratory and field cage tests, and could improve the efficacy of operational medfly SIT programs.

### Example 11

### Field Cage Tests for Mating Competitiveness

Males from line #67 (non-irradiated or irradiated with 120 Gy, 48 hours before adult emergence) were competed against non-irradiated wild type Argentinean males for mating with Argentinean wild-type females in a field cage. Pupae from the different strains/treatment were placed in emergence cages, and every 24 h adults were removed, sorted by sex, and placed in cages with adult food (3:1, sugar:hydrolyzed yeast) and water for 6 d. Two days before the tests, flies were marked with a dot of water-based paint on the thorax (DEKA®, Unterhaching, Germany). In each field cage, three potted Citrus aurantius trees, 1.6 m in height with 1.5-m-diameter canopy, were used as a mating arena. To follow the quarantine protocol, tests were performed in a greenhouse with controlled temperature (24-26° C.) and humidity (60-80%). On the day of the test, 20 sexually mature non-irradiated Argentinean males, 20 non-irradiated and 20 irradiated males from line #67 were released into the cage around 08:30. Approximately 20 min later, 20 virgin and sexually mature Argentinean females were released in the cage. Tests lasted 3 hours. Mating pairs were collected as they formed by allowing the pair to walk into a small vial. The type of mating couple was recorded and the proportion of mating was calculated for each mating type (see FIG. 8). After the couples separated, the males and females were identified and the mated females were grouped together depending on the type of mated male and transferred to small egging cages. Eggs were collected for five consecutive days and transferred to small Petri dishes with moist black filter paper. After four days of incubation, hatched larvae and un-hatched eggs were counted to determine the egg hatch for each mating type. Twelve replications of this test were carried out.

### Example 12

### Tc Dependent Reversible Lethality

To test the reversibility of the embryonic lethality, three-day old flies from lines #66 or #67 were transferred from Tc-containing (10 μg/ml) to Tc-free adult medium. After five days the flies were transferred back to Tc-containing (10 μg/ml) adult medium and reared for additional 3 days. Progeny of 24 h egg lay intervals over the complete period were monitored (embryos from Tc-containing or Tc-free adult medium were reared on 1 μg/ml Tc-containing or Tc-free larval food, respectively; eggs and adults were scored). Two independent time series were performed for both transgenic lines.

After transfer to Tc-free medium the rate of progeny decreased in five days to 0%. The sterility could be reversed by retransfer of the adults to Tc-containing medium (FIG. 5C). The reduced rate of progeny after this procedure could be due to a slight irreversible effect of the lethal system or to the age of flies as shown in other studies (SCOLARI (2008)).

TABLE 1
Statistical analysis.
(A) T-test for the efficiency tests

| | | stat | df | probability | significance |
|---|---|---|---|---|---|
| LL #29 | L1 larvae | 4.9448 | 2 | 0.1270 | ns |
| | pupae | 0.1624 | 2 | 0.8975 | ns |
| | adults | 0.1177 | 2 | 0.9254 | ns |
| LL #72 | L1 larvae | 2.2391 | 2 | 0.2673 | ns |
| | pupae | 0.8260 | 2 | 0.5604 | ns |
| | adults | 1.4728 | 2 | 0.3797 | ns |
| LL #66 | L1 larvae | 1.6379 | 2 | 0.3489 | ns |
| | pupae | — | — | — | — |
| | adults | — | — | — | — |
| LL #67 | L1 larvae | — | — | — | — |
| | pupae | — | — | — | — |
| | adults | — | — | — | — |
| LL #68 | L1 larvae | 0.6821 | 2 | 0.6188 | ns |
| | pupae | 0.2245 | 2 | 0.8593 | ns |
| | adults | — | — | — | — |
| WT | L1 larvae | 1.6911 | 2 | 0.3399 | ns |
| | pupae | 1.3158 | 2 | 0.4137 | ns |
| | adults | 1.9443 | 2 | 0.3024 | ns |

(B) T-test for the competition tests

| | stat | df | probability | significance |
|---|---|---|---|---|
| + | 0.9017 | 10 | 0.4182 | ns |
| 66 (1:1:1) | 0.7000 | 10 | 0.5151 | ns |
| 66 (1:1:3) | 1.0101 | 10 | 0.3588 | ns |
| 66 (1:1:5) | 1.4861 | 10 | 0.1974 | ns |
| 66 (1:1:9) | 1.0613 | 10 | 0.3371 | ns |
| 67 (1:1:1) | 0.1265 | 10 | 0.9043 | ns |
| 67 (1:1:3) | 0.0690 | 10 | 0.9477 | ns |
| 67 (1:1:5) | 1.7320 | 10 | 0.1438 | ns |
| 67 (1:1:9) | 1.4029 | 10 | 0.2196 | ns |
| ++ | 0.0987 | 10 | 0.9261 | ns |

(C) Chi-test for the reversibility tests

| | stat | df | probability | significance |
|---|---|---|---|---|
| Day 1 (+Tc) | 0.0008 | 1 | 0.9768 | ns |
| Day 2 (+Tc) | 0.0067 | 1 | 0.9348 | ns |
| Day 3 (−Tc) | 0.0152 | 1 | 0.9020 | ns |
| Day 4 (−Tc) | 0.0098 | 1 | 0.9213 | ns |
| Day 5 (−Tc) | 0.0120 | 1 | 0.9127 | ns |
| Day 6 (−Tc) | 0.0139 | 1 | 0.9060 | ns |
| Day 7 (−Tc) | — | — | — | ns |
| Day 8 (+Tc) | 0.0314 | 1 | 0.8593 | ns |
| Day 9 (+Tc) | 0.0088 | 1 | 0.9251 | ns |
| Day 10 (+Tc) | 0.0059 | 1 | 0.9389 | ns |

ns = non-significantly different; — = the original data was 0 for all repetitions, statistics are therefore not possible.

### LIST OF REFERENCES

Bergmann, A., Agapite, J., McCall, K. and Steller, H. The Drosophila gene hid is a direct molecular target of Ras-dependent survival signaling. Cell 1998; 95: 331-341.
Cary L. C., Goebel M, Corsaro B. G., Wang H. G., Rosen E., Fraser M. J. Transposon mutagenesis of baculoviruses: analysis of Trichoplusia ni transposon IFP2 insertions within the FP-locus of nuclear polyhedrosis viruses. Virology 1989; 172(1):156-69.
Condon K. C., Condon G. C., Dafa'alla T. H., Fu G., Phillips C. E., Jin L., Gong P., Alphey L. Genetic sexing through the use of Y-linked transgenes. Insect Biochem Mol Biol 2007; 37(11):1168-76.
Davis, G. K., Jaramillo, C. A. and Patel, N. H. Pax group III genes and the evolution of insect pair-rule patterning. Development 2001; 128: 3445-3458.
Franz, G. Genetic sexing strains in Mediterranean fruit fly, an example for other species amenable to large-scale rearing for the sterile insect technique. In Sterile Insect Technique—Principles and Practice in Area-Wide Integrated Pest Management 2005; V. A. Dyck, J. Hendrichs and A. S. Robinson, ed., Dordrecht, Springer: 427-451.
Franz, G. (2000). The “Combi Fly Concept” revisited: how much radiation is required to sterilise males of a genetic sexing strain? In Area-wide control of fruit flies and other insect pests (ed. K. H. Tan), pp. 511-516. Pulau Pinang, Malaysia: Penerbit Universiti Sains Malaysia.
Fu, G., Condon, K. C., Epton, M. J., Gong, P., Jin, L., Condon, G. C., Morrison, N. I., Dafa'alla, T. H. and Alphey, L. Female-specific insect lethality engineered using alternative splicing. Nature Biotechnology 2007; 25: 353-357.
Gariou-Papalexiou, A., et al. Polytene chromosomes as tools in the genetic analysis of the Mediterranean fruit fly. Ceratitis capitata. Genetica 2002; 116: 59-71.
Gong, P., Epton, M. J., Fu, G., Scaife, S., Hiscox, A., Condon, K. C., Condon, G. C., Morrison, N. I., Kelly, D. W., Dafa'alla, T. et al. A dominant lethal genetic system for autocidal control of the Mediterranean fruitfly. Nature Biotechnology 2005; 23: 453-456.
Groth, A. C., Fish, M., Nusse, R. and Calos, M. P. Construction of transgenic Drosophila by using the site-specific integrase from phage phiC31. Genetics 2004; 166: 1775-1782.
Handler, A. M. and Harrell, R. A. Germline transformation of Drosophila melanogaster with the piggyBac transposon vector. Insect Molecular Biology 1999; 8: 449-457.
Handler, A. M. and Harrell, R. A. Polyubiquitin-regulated DsRed marker for transgenic insects. Biotechniques 2001a; 31: 820-828.
Handler, A. M. and Harrell, R. A. Transformation of the Caribbean fruit fly, Anastrepha suspensa, with a piggyBac vector marked with polyubiquitin-regulated GFP. Insect Biochemistry and Molecular Biology 2001b; 31: 199-205.
Handler, A. M. and James, A. A. Insect Transgenesis: Methods and Applications 2000; Boca Raton, CRC Press LLC.
Horn, C., Schmid, B. G. M., Pogoda, F. S. and Wimmer, E. A. Fluorescent transformation markers for insect transgenesis. Insect Biochemistry and Molecular Biology 2002; 32: 1221-1235.
Horn, C. and Wimmer, E. A. A transgene-based, embryo-specific lethality system for insect pest management. Nature Biotechnology 2003a; 21: 64-70.
Horn, C. and Wimmer, E. A. A versatile vector set for animal transgenesis. Development Genes and Evolution 2000; 210: 630-637.
Horn, C., Offen, N., Nystedt, S., Hacker, U. and Wimmer, E. A. piggyBac-Based Insertional Mutagenesis and Enhancer Detection as a Tool for Functional Insect Genomics. Genetics 2003b; 163: 647-661.
McGuire S. E., Roman G., Davis R. L. Gene expression systems in Drosophila: a synthesis of time and space. Trends Genet 2004; 20(8):384-91.
Niyazi, N., Caceres, C., Delprat, A., Wornoayporn, V., Ramirez Santos, E., Franz, G. and Robinson, A. S. Genetics and mating competitiveness of Ceratitis capitata (Diptera: Tephritidae) strains carrying the marker sergeant, sr². Annals of the Entomology Society of America 2005; 98: 119-125.
Parker, A. and Mehta, K. (2007). Sterile insect technique: a model for dose optimization for improved sterile insect quality. Florida Entomologist 90, 88-95.
Rong, Y. S., Titen S. W., Xie H. B., Golic M. M., Bastiani M., Bandyopadhyay P., Olivera B. M., Brodsky M., Rubin G. M., and Golic K. G. Targeted mutagenesis by homologous recombination in D. melanogaster. Genes Dev 2002; 16(12):1568-81.
Sarkar, A., et al. Insulated piggyBac vectors for insect transgenesis. BMC Biotechnol. 2006; 6: 27.
Saul, S. H. Rosy-like mutant of the Mediterranean fruit fly, Ceratitis capitata (Diptera: Tephritidae), and its potential for use in a genetic sexing program. Annals of the Entomological Society of America 1982; 75: 480-483.
Schetelig, M. F., Horn, C., Handler, A. M. and Wimmer, E. A. Development of an embryonic lethality system for SIT in Ceratitis capitata. In Area-wide control of insect pests: from research to field implementation 2007; M. J. B. Vreysen, A. S. Robinson und J. Hendrichs, ed., Dordrecht, Netherlands: Springer: 85-93.
Scolari, F., et al. Fluorescent sperm marking to improve the fight against the pest insect Ceratitis capitata (Wiedemann; Diptera: Tephritidae). New Biotech 2008; in press.
Thomas, D. D., Donnelly, C. A., Wood, R. J. und Alphey, L. S. Insect population control using a dominant, repressible, lethal genetic system. Science 2000; 287: 2474-2476.
Thorpe, H. M., Wilson, S. E. and Smith, M. C. Control of directionality in the site-specific recombination system of the Streptomyces phage phiC31. Molecular Microbiology 2000; 38: 232-241.
Wimmer, E. A. Insect transgenesis by site-specific recombination. Nature Methods 2005; 2: 580-582
Zacharopoulou, A., et al. The genome of the Mediterranean fruitfly Ceratitis capitata: localization of molecular markers by in situ hybridization to salivary gland polytene chromosomes. Chromosoma 1992; 101: 448-455.

1-20. (canceled)

21. A transgenic insect comprising a developmental stage-specific lethality system comprising
a) a first gene expression cassette comprising, in operative linkage,
(i) a first promoter/enhancer element of a developmental stage-specific gene derived from a insect pest species, or a functional derivative of said promoter/enhancer element,
preferably wherein the promoter/enhancer element or functional derivative thereof is derived from a member of the family Tephritidae,
more preferably wherein the promoter/enhancer element is selected from the group consisting of the promoter/enhancer element of the C.c.-serendipity α gene (SEQ ID NO. 1) or a functional derivative thereof, the promoter/enhancer element of the C.c.-CG2186 gene (SEQ ID NO. 2) or a functional derivative thereof, the promoter/enhancer element of the C.c.-slow as molasses gene (SEQ ID NO. 3) or a functional derivative thereof, the promoter/enhancer element of the C.c.-sub2_{—}99 gene (SEQ ID NO. 4) or a functional derivative thereof, the promoter/enhancer element of the C.c.-sub2_{—}63 gene (SEQ ID NO. 5) or a functional derivative thereof, and the promoter/enhancer element or a functional derivative thereof of the C.c.-sub2_{—}65 gene, the gene having SEQ ID NO. 6,
(ii) a first component of a transactivating system, whose activity is controllable by a suitable exogenous factor,
and
b) a second gene expression cassette comprising, in operative linkage,
(i) a second component of the transactivating system,
(ii) a second promoter that is responsive to the activity of the transactivating system, and
(iii) a lethality inducing component.

22. The transgenic insect of claim 21, wherein the activity of the transactivating system can be repressed by the presence of the exogenous factor.

23. The transgenic insect of claim 21, wherein the first promoter/enhancer element is selected from the group of the promoter/enhancer element of the C.c.-serendipity α gene (SEQ ID NO. 1) and the promoter/enhancer element of the C.c.-CG2186 gene (SEQ ID NO. 2), or a functional derivative thereof,
preferably wherein the promoter/enhancer element is the promoter/enhancer element of the C.c.-serendipity α gene (SEQ ID NO. 1) or a functional derivative thereof.

24. The transgenic insect of claim 21, wherein the second promoter is selected from the group consisting of the Drosophila melanogaster hsp70 basal promoter (SEQ ID NO. 14) and the Drosophila melanogaster P basal promoter (SEQ ID NO: 15), or a functional derivative thereof,
preferably wherein the second promoter is the Drosophila melanogaster hsp70 basal promoter (Seq ID NO: 14) or a functional derivative thereof.

25. The transgenic insect of claim 21, wherein the first component of the transactivating system is capable of expressing the tetracycline-repressible transactivator (tTA) (SEQ ID NO: 16) or a functional derivative thereof, the second component of the transactivating system comprises a tTA-responsive element, and the suitable exogenous factor is tetracycline, or a functional derivative or functional analogue thereof.

26. The transgenic insect of claim 21, wherein the lethality inducing system is selected from the group consisting of a pro-apoptotic gene, an apoptotic gene, toxins, hyperactive cell signalling molecules, and RNAi,
preferably wherein lethality inducing system comprises the gene hid^{Ala5} or a functional derivative thereof.

27. The transgenic insect of claim 21, wherein the first and/or the second gene expression cassette further comprises a minimal attachment P (attP) site (SEQ ID NO: 17), or a functional derivative thereof.

28. The transgenic insect of claims 21, wherein the insect is homozygous for the first and/or the second gene expression cassette.

29. The transgenic insect of claim 21, wherein further each of the first and/or the second gene expression cassette are comprised in a suitable vector construct,
preferably wherein the vector construct further comprises one or more elements selected from the group consisting of a transposon, a polytropic transposon, a retrovirus, a polytropic retrovirus, an element capable of homologous recombination, and an element capable of non-homologous recombination.

30. The transgenic insect of claims 21, wherein the insect belongs to the family Tephritidae, preferably to the genus Ceratitis, and most preferably to the species Ceratitis capitata.

31. The transgenic insect of claim 30, wherein the first gene expression cassette and/or the second gene expression cassette is/are located on chromosome 5 of Ceratitis capitata,
preferably wherein the first gene expression cassette and the second gene expression cassette are both located on chromosome 5 of Ceratitis capitata.

32. The transgenic insect of claim 31, wherein the first or second gene expression cassette is located at a position selected from the group consisting of position 70B and position 63B of chromosome 5 of Ceratitis capitata.

33. The transgenic insect of claims 32, wherein the first gene expression cassette is located at or near the nucleic acid sequence of SEQ ID NO. 13 of chromosome 5 of Ceratitis capitata, preferably wherein the first gene expression cassette is located at the nucleic acid sequence “ttaa” at position 85-88 of SEQ ID NO. 13 of chromosome 5 of Ceratitis capitata,
and/or wherein the second gene expression cassette is located at or near the nucleic acid of sequence SEQ ID NO. 12 of chromosome 5 of Ceratitis capitata, preferably wherein the second gene expression cassette is located at the nucleic acid sequence “ttaa” at position 178-181 of SEQ ID NO. 12 of chromosome 5 of Ceratitis capitata.

34. A method of controlling reproduction in an insect population of interest, comprising the steps of:
(i) providing a plurality of insects according to claim 1 capable of interbreeding with the insects of the population of interest,
(ii) optionally selecting suitable individual insects from the plurality, and
(iii) allowing the insects of step (i) or (ii) to interbreed with insects of the population of interest,
optionally wherein the insects of step (i) or (ii) are released in an area where reproduction control of insects of the population of interest is desirable.

35. The method of claim 34, wherein in step (i), insects are provided that further comprise a sexing system, preferably a genetic sexing system, and/or wherein in step (ii), male insects are selected from the plurality.

36. A method for producing transgenic insects comprising a developmental stage-specific lethality system, comprising the steps of:
(i) providing a set of insects comprising a first gene expression cassette and/or a second gene expression cassette according to claim 1,
(ii) optionally subjecting the set of insects to one or more steps of interbreeding,
(iii) evaluating the set of insects of (i) or offspring obtained from the interbreeding steps of (ii) for functionality of the developmental stage-specific lethality system,
optionally wherein the insects of step (i) or (ii) are released in an area where reproduction control of insects of the population of interest is desirable.

37. The method of claim 36, wherein the transgenic insects are as defined in claim 1, preferably wherein the transgenic insects are pest insects.

38. Method of using a transgenic insect according to claim 1 for controlling reproduction in an insect population of interest, wherein the transgenic insect is capable of interbreeding with insects of the population of interest.

39. A developmental stage-specific lethality system for use in a transgenic insect, comprising
(i) a first gene expression cassette according to claim 1, and
(ii) a second gene expression cassette according to claim 1.

40. The system of claim 39, wherein the transgenic insect is as defined in claim 1.
